(19) United States
(12) Patent Application Publication
(10) Pub. No.: US 2012/0174920 A1
(43) Pub. Date: Jul. 12, 2012

(54) Multi-Layer Endotracheal Tube Apparatus

(75) Inventors: Nir Barkai, Kfar Saba (IL); Mark Shahar, Holon (IL)

(73) Assignee: Tubular Medical Ltd., Kfar Saba (IL)

(21) Appl. No.: 13/381,259

(22) PCT Filed: Jul. 1, 2010
(86) PCT No.: PCT/IL10/00531

§ 371 Date: Mar. 23, 2012

Related U.S. Application Data
(60) Provisional application No. 61/222,540, filed on Jul. 2, 2009.

(51) Int. Cl. A61M 16/04

(52) U.S. Cl. 128/200.26

(57) ABSTRACT

Multi layer endotracheal tube apparatus (800) comprising an outer flexible elongated tube layer (802) having a proximal end (816) and a distal end (818), defining an inner surface (820) therebetween, at least one flexible elongated inner tube layer (804) having a proximal end (822) and a distal end (824), defining an inner surface (828), the distal end (824) of the inner tube layer (804) defining a distal port section (830), the inner tube layer (804) extending along inner length of the outer tube layer (802), the distal end (824) of the inner tube layer (804) detachably coupled along a closed circumference of the inner surface (820) of the outer tube layer (802), and at least one closure mechanism (806) operative to close the distal port section (830), and detach the distal end (824) of the inner tube layer (804) from the inner surface (820) of the outer tube layer.

### FIELD OF THE DISCLOSED TECHNIQUE

The disclosed technique relates to medical devices, in general, and to endotracheal tube apparatuses and methods for reducing risks of acquiring medical complications associated with endotracheal intubation and tracheostomy, in particular.

### BACKGROUND OF THE DISCLOSED TECHNIQUE

Endotracheal intubation is a procedure where one end of an endotracheal tube (ETT), typically a flexible tube, is inserted through the mouth or nose into the trachea (i.e., the windpipe), left in-situ in order to maintain an unobstructed passageway (e.g., an airway) particularly for delivery of oxygen, anesthesia and medication to the lungs. The ETT, also called a breathing tube, is further employed during mechanical ventilation, as well as to permit suctioning of mucus, in order to prevent a build-up of secretions that can cause blockage of the airway. Similarly, tracheostomy is an invasive surgical procedure involving the insertion of a tracheostomy tube through an incision in the trachea. The other end of the ETT or the tracheostomy tube is usually connected to a mechanical ventilator (e.g., a breathing machine) or to a manual ventilator (e.g., a manual resuscitator, a bag valve mask, a continuous-flow breathing bag), both types of which function to provide oxygen-gas mixture to the lungs. Mechanical ventilation is a method of artificial ventilation of the lungs, typically employed after invasive intubation, in order to mechanically assist in breathing, in settings such as in the intensive care unit (ICU). Although endotracheal intubation is regarded as one of the most reliable methods for airway management, it does not entirely preclude potential problems and risks associated with this intervention. Among the various medical complications associated with the use of endotracheal tubes, several of which include, for example, lobar collapse, hypoxemia, hypercapnoea, tube blockage and localized trauma to the airway, there is a condition called ventilator-associated pneumonia (VAP).

VAP is a nosocomial (i.e., hospital-acquired) pneumonia which occurs in those who are on mechanical ventilation via an endotracheal or tracheostomy tube, typically for a period of at least 48 hours. The primary routes of acquiring (endemic) VAP is from microorganisms such as by oropharyngeal colonization of endogenous flora and by pathogens acquired exogenously from the ICU environment. The endotracheal and tracheostromy tubes allow passage of microorganisms (e.g., bacteria) from the ICU environment (e.g., contaminated respiratory equipment, contaminated air) into the lower respiratory tract (e.g., the alveoli). VAP may typically develop from aspiration of microbe-laden secretions from the oropharynx, or indirectly, by reflux from the stomach into the lower respiratory tract via the oropharynx. Moreover, microorganisms colonize and build-up on the inner, as well as the outer surface of the endotracheal tube, and are embolized into the lungs during the intake of air. Furthermore, is has been implicated that biofilm formation on the inner and outer surfaces of endotracheal tubes is an important promoter of bacterial colonization of the lower respiratory tract and as a cause of VAP. In addition, a build-up of secretions and other accumulated substances (e.g., viscous medications administered as powders or emulsions) on the inner surface of the endotracheal tube progressively decrease the internal volume of the tube available for respiration, thereby increasing resistance to airflow, thus reducing airflow capacity and increasing the peak inspiratory pressure (PIP).

Techniques for reducing the risk of acquiring VAP and other medical conditions associated with endotracheal and tracheostomy tubes are known in the art. An article by Oslon, Merle E. et al., entitled “Silver-Coated Endotracheal Tubes Associated with Reduced Bacterial Burden in the Lungs of Mechanically Ventilated Dogs” in CHEST—Official Journal of the American College of Chest Physicians 121:3 (2002):863-870 is directed to a study to evaluate the influence of silver-coated endotracheal tubes on the lung bacterial burden of mechanically ventilated dogs. The study included endotracheal tubes, which both their inner and outer surfaces were coated with an antimicrobial silver-hydrogel formulation. According to the results of this study, the silver coating of the endotracheal tubes may delay the onset of and decrease the severity of aerobic bacteria colonization in the lungs.

U.S. Pat. No. 7,258,120 B1 to Melker, entitled “Endotracheal Tube Apparatus and Method for Using the Same to Reduce the Risk of Infections” is directed to an endotracheal tube apparatus having a tube within a tube arrangement. The endotracheal tube apparatus includes a first elongated tube, a second elongated tube, a first connector, and a second connector. The first elongated tube and the second elongated tube each include a proximal end a distal end, an inner and an outer wall. The first tube is inserted inside the length of the second elongated tube. The second connector includes two ports. One of the ports is attached to the proximal end of the second tube and the other port is attached to a suction apparatus. The first connector includes two ports, one port attached to the proximal end of the first tube and another tube attached to a ventilation device. The first connector is attached from the second connector during mechanical ventilation of a patient. Either the outer wall of the first tube or the inner wall of the second tube includes raised structures, which form a first suction channel throughout the length of the apparatus between the elongated tubes.

The first suction channel allows suction while the patient is intubated in order to remove gases and secretions. At a predetermined time, the first elongated tube is removed, cleaned, and replaced with another first elongated tube while the patient is still intubated with the second elongated tube.

PCT International Publication No. WO 2005/018713 A2 by Angel, entitled “Airway Assembly for Tracheal Intubation” is directed to an airway assembly to be used in situations for those requiring assistance in breathing. The airway assembly includes a first conduit, an elongated member, a stent, and a sleeve. The first conduit includes a proximal end and a distal end. The first conduit is positioned in the sleeve. The sleeve is removably coupled to the first conduit. The elongated member is positionable in the first conduit. The stent is coupled toward the distal end of the first conduit. The airway assembly is inserted in an air passage of a patient. The proximal end is coupled to a supply line. The first conduit functions to deliver air to the body lumen and consequently to the patient. The supply line allows fluid to be inserted into a region beyond the distal end of the airway assembly. The sleeve functions to inhibit a stent from expanding. The sleeve is configured to peel away from the first conduit after the airway assembly has been inserted into a body lumen. The stent functions to inhibit the body lumen from collapsing.

### SUMMARY OF THE DISCLOSED TECHNIQUE

It is the object of the disclosed technique to provide a novel multi-layer endotracheal tube apparatus that employs an outer flexible elongated tube layer that is generally hollow, a plurality of flexible elongated inner tube layers that extend substantially along and within the outer flexible elongated tube layer, and a plurality of closure mechanisms that are each operative to close a distal port section of a respective flexible elongated inner tube layer. Each of the flexible elongated inner tube layers are layered on each other and inside the outer flexible elongated tube layer so as to form an a multi-layer endotracheal tube. Each closure mechanism is operative to seal biological material (e.g., secretions, pathogens, microorganisms), accumulated within the internal volume of its respective flexible elongated inner tube layer, such that substantially no biological material leaks out therefrom, while the each flexible elongated inner tube layer is removed. In this manner, substantially no bacterial residue is left behind during the removal process. When a flexible elongated inner tube layer, which has been exposed to biological material, is removed, a new flexible inner tube layer is revealed underneath, and the process is repeated until all of the flexible elongated inner tube layers are removed.

In accordance with the disclosed technique, there is thus provided a multi-layer endotracheal tube apparatus including an outer flexible elongated tube layer, at least one flexible elongated inner tube layer, and at least one closure mechanism. The outer flexible elongated tube layer has a proximal end and a distal end which define an inner surface therebetween. The flexible elongated inner tube layer has a proximal end and a distal end, which define an inner surface, and outer surface and an internal volume therebetween. The distal end of the flexible inner tube layer defines a distal port section. The flexible elongated inner tube layer extends substantially within and at least partially along inner length of the outer flexible elongated tube layer. The distal end of the flexible elongated inner tube layer is detachably coupled along an inner substantially closed circumference of the inner surface of the outer flexible elongated tube layer. The closure mechanism is at least partially located in an area of the distal end of the flexible elongated inner tube layer. The closure mechanism is operative to close the distal port section and detach the distal end of the flexible elongated inner tube layer from the inner surface of the outer flexible elongated tube layer, as to enable withdrawal of the flexible elongated inner tube layer via the proximal end of the outer flexible elongated tube layer.

According to another aspect of the disclosed technique, there is thus provided a method removal of an inner layer from within a multi-layer endotracheal tube, and material substantially contained within a volume defined by the inner layer. The multi-layer endotracheal tube includes an outer flexible elongated tube layer, at least one flexible elongated inner tube layer and a closure mechanism. The outer flexible elongated tube layer has a proximal end and distal end, which define an inner surface therebetween. The flexible elongated inner tube layer has a proximal end and a distal end that defines a distal port section. The flexible elongated inner tube layer extends substantially within and at least partially along inner length of the outer flexible elongated tube layer. The distal end of the flexible elongated inner tube layer detachably coupled along an inner substantially closed circumference of the inner surface of the outer flexible elongated tube layer. The closure mechanism is at least partially located in an area of the distal end of the flexible elongated inner tube layer. The method includes the procedures of closing the distal port section by the closure mechanism, detaching the distal end of the flexible elongated inner tube layer from the inner surface of the outer flexible elongated tube layer, and removing the flexible elongated inner tube layer from within the outer flexible elongated tube layer via the proximal end of the outer flexible elongated tube layer.

According to a further aspect of the disclosed technique, there is thus provided a multi-layer endotracheal tube apparatus including an outer flexible elongated tube layer and at least one flexible inner tube layer. The outer flexible elongated tube layer has a proximal end and a distal end, which define an inner surface therebetween. The flexible inner tube layer has a proximal end and a distal end. At least part of the flexible inner tube layer is layered onto the inner surface of the outer flexible tube layer. The flexible inner tube layer extends substantially within and at least partially along inner length of the outer flexible elongated tube layer. The flexible inner tube layer is operative to detach from within the outer flexible elongated inner tube layer, as to enable withdrawal of the flexible inner tube layer via the proximal end of the outer flexible elongated tube layer.

According to another aspect of the disclosed technique, there is thus provided a multi-layer endotracheal tube apparatus that includes an outer flexible elongated tube layer and a flexible elongated layer. The flexible elongated tube layer has a proximal end and a distal end, which define an inner surface and an outer surface therebetween. The flexible elongated layer has an engaging surface and an exposure surface. The flexible elongated layer extends substantially from the proximal end and substantially along the outer surface toward the distal end where the flexible elongated layer continues to extend into the outer flexible elongated tube layer substantially along the inner surface toward the proximal end, so as to enable withdrawal of at least part of the flexible elongated layer via the proximal end, such that only the engaging surface of the flexible elongated layer is in contact with the inner surface and the outer surface.

### BRIEF DESCRIPTION OF THE DRAWINGS

The disclosed technique will be understood and appreciated more fully from the following detailed description taken in conjunction with the drawings in which:

FIG. 1A is a schematic illustration of a multi-layer endotracheal tube apparatus, constructed and operative in accordance with an embodiment of the disclosed technique;

FIG. 1B is a schematic illustration depicting an operative state of the multi-layer endotracheal tube apparatus of FIG. 1A;

FIG. 1C is a schematic illustration depicting another operative state of the multi-layer endotracheal tube apparatus of FIG. 1A;

FIG. 2 is a schematic illustration of a partially collapsible flexible elongated inner tube layer, constructed and operative in accordance with an alternative embodiment to the disclosed technique of FIGS. 1A, 1B, and 1C;

FIG. 3A is a schematic illustration of a multi-layer endotracheal tube apparatus, utilizing an inflatable closure mechanism, constructed and operative in accordance with another alternative embodiment to the disclosed technique of FIGS. 1A, 1B, and 1C;

FIG. 3B is a schematic illustration depicting the multi-layer endotracheal tube apparatus of FIG. 3A in a particular operative state;

FIG. 4A is a schematic illustration of a multi-layer endotracheal tube apparatus, having an inner tube layer that is at least partially constructed to crumple, constructed and operative in accordance with another alternative embodiment of the disclosed technique;

FIG. 4B is a schematic illustration of the multi-layer endotracheal tube apparatus of FIG. 4A in a particular operative state;

FIG. 4C is a schematic illustration of the multi-layer endotracheal tube apparatus of FIG. 4A in another operative state;

FIG. 4D is a schematic illustration of the multi-layer endotracheal tube apparatus of FIG. 4A in a further operative state;

FIG. 5A is a schematic illustration of a multi-layer endotracheal tube apparatus, generally referenced 500, having an inner tube layer that is operative to roll close, constructed and operative in accordance with a further alternative embodiment of the disclosed technique;

FIG. 5B is a schematic illustration of the multi-layer endotracheal tube apparatus of FIG. 5A in a particular operative state;

FIG. 5C is a schematic illustration of the multi-layer endotracheal tube apparatus of FIG. 5A in another operative state;

FIG. 6A is a schematic illustration of a multi-layer endotracheal tube apparatus of hybrid construction, constructed and operative according to another embodiment of the disclosed technique;

FIG. 6B is a schematic illustration depicting an operative state of the multi-layer endotracheal tube apparatus of FIG. 6A;

FIG. 6C is a schematic illustration depicting another operative state of the multi-layer endotracheal tube apparatus of FIG. 6A;

FIG. 7A is a schematic cross-sectional illustration in longitudinal section view of a multi-layer endotracheal tube apparatus, having a parallel inner tube layer stack, constructed and operative in accordance with a further embodiment of the disclosed technique;

FIG. 7B is a schematic cross-sectional illustration in longitudinal section view of the multi-layer endotracheal tube apparatus of FIG. 7A in a particular operative state;

FIG. 7C is a schematic cross-sectional illustration in longitudinal section view of the multi-layer endotracheal tube apparatus of FIG. 7A in another operative state;

FIG. 7D is a schematic cross-sectional illustration in longitudinal section view of a multi-layer endotracheal tube apparatus, having a perpendicular inner tube layer stack, constructed and operative in accordance with another embodiment of the disclosed technique;

FIG. 7E is a schematic cross-sectional illustration in longitudinal section view of the multi-layer endotracheal tube apparatus of FIG. 7D in a particular operative state;

FIG. 7F is a schematic cross-sectional illustration in longitudinal section view of a multi-layer endotracheal tube apparatus, having inner tube layers that peel from within the outer tube layer, constructed and operative in accordance with a further embodiment of the disclosed technique;

FIG. 7G is a schematic cross-sectional illustration in longitudinal section view of the multi-layer endotracheal tube apparatus of FIG. 7F in a particular operative state;

FIG. 7H is a schematic cross-sectional illustration in longitudinal section view of a multi-layer endotracheal tube apparatus, employing external-body inner layer dispensation, constructed and operative in accordance with another embodiment of the disclosed technique;

FIG. 7I is a schematic cross-sectional illustration in longitudinal section view of the multi-layer endotracheal tube apparatus of FIG. 7H in a particular operative state;

FIG. 7J is a schematic cross-sectional illustration in longitudinal section view of the multi-layer endotracheal tube apparatus of FIG. 7H in another operative state;

FIG. 8A is a schematic illustration of a multi-layer endotracheal tube apparatus including a laryngeal mask portion, generally referenced 800, constructed and operative in accordance a further embodiment of the disclosed technique;

FIG. 8B is a schematic illustration of the multi-layer endotracheal tube apparatus including the laryngeal mask portion of FIG. 8A, in a particular operative state;

FIG. 8C is a schematic illustration of the multi-layer endotracheal tube apparatus including the laryngeal mask portion of FIG. 8A, in another operative state; and

FIG. 8D is a schematic illustration of the multi-layer endotracheal tube apparatus including the laryngeal mask portion of FIG. 8A, in a further operative state.

### DETAILED DESCRIPTION OF THE EMBODIMENTS

The disclosed technique overcomes the disadvantages of the prior art by providing a multi-layer endotracheal tube apparatus employing an outer flexible elongated tube layer that is generally hollow, a plurality of flexible elongated inner tube layers that extend substantially along and within the outer flexible elongated tube layer, and a plurality of closure mechanisms, each of which is operative to close a distal port section of a respective flexible elongated inner tube layer. Each closure mechanism is operative to seal biological material (e.g., secretions, pathogens, microorganisms, and the like), accumulated within the internal volume of the respective flexible elongated inner tube layer, such that substantially no biological material leaks out therefrom, while the flexible elongated inner tube layer is removed. In this manner, substantially no bacterial residue is left behind during the removal process. Each flexible elongated inner tube layer is associated (i.e., paired) with a respective closure mechanism so when a flexible elongated inner tube layer, which has been exposed to biological material, is removed with its respective closure mechanism, a new pair is revealed underneath, and the process is repeated.

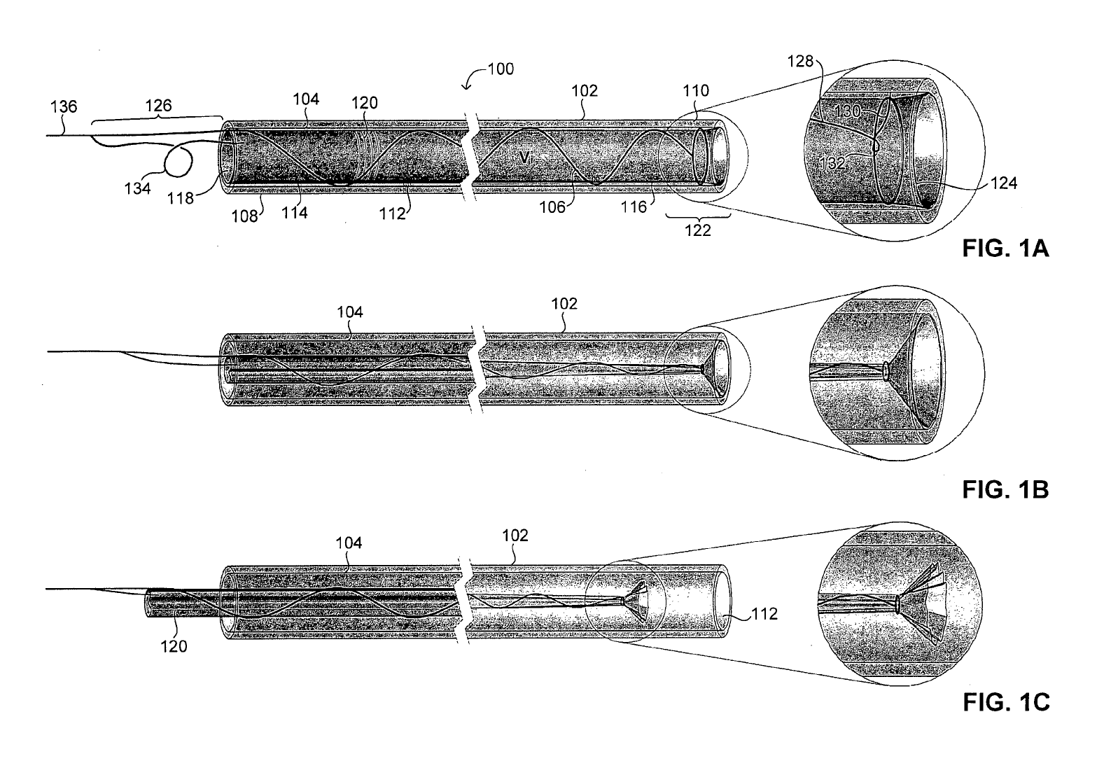

Reference is now made to FIGS. 1A, 1B and 1C. FIG. 1A is a schematic illustration of a multi-layer endotracheal tube apparatus, generally referenced 100, constructed and operative in accordance with an embodiment of the disclosed technique. FIG. 1B is a schematic illustration depicting an operative state of the multi-layer endotracheal tube apparatus of FIG. 1A. FIG. 1C is a schematic illustration depicting another operative state of the multi-layer endotracheal tube apparatus of FIG. 1A. Multi-layer endotracheal tube apparatus 100 (FIG. 1A) includes an outer flexible elongated tube layer 102, a plurality (not shown) of inner tube layers, where at least one of these layers is shown as collapsible flexible elongated inner tube layer 104, and a closure mechanism 106. Although not limited to one inner tube layer, multi-layer endotracheal tube apparatus 100 will be described in conjunction with the accompanying figures as having one inner tube layer, for the purposes of elucidating the disclosed technique.

Outer flexible elongated tube layer 102 is substantially tubular and has a proximal end 108, and a distal end 110, both of which define an inner surface 112 therebetween. Distal end 110 may be beveled (not shown). Collapsible flexible elongated inner tube layer 104 has a proximal end 114 and a distal end 116, which defines an inner surface 118, and an outer surface 120 (shown more distinctly in FIG. 1C) therebetween. The volume substantially contained within inner surface 118 defines a non-zero internal volume of multi-layer endotracheal tube apparatus 100. Distal end 116 defines a distal port section 122, which in turn is terminated by rim 124. Collapsible flexible elongated inner tube layer 104 extends substantially within, and along the inner length of outer flexible elongated tube layer 102, as shown in FIG. 1A. Rim 124 of distal end 116 is detachably coupled (e.g., adhered by glue) along an inner substantially closed circumference (e.g., circular, elliptical) of inner surface 112 of outer flexible elongated tube layer 102. This circumferential coupling of rim 124 with inner surface 112 isolates (i.e., separates) a portion of inner surface 112 that which extends from the point of coupling toward proximal end 108, from fluids (e.g., secretions, air), which enter via distal end 110. Thus, the circumferential coupling of rim 124 with inner surface 112 ensures that inner surfaces of intermediate tube layers (not shown), sandwiched between outer flexible elongated tube layer 102 and collapsible flexible elongated inner tube layer 104, are not exposed to contamination, pathogens, bacteria, accumulation of secretions, and the like. Closure mechanism 106 (i.e., embodied according to this embodiment of the disclosed technique as a string (hereinafter referred, in this embodiment as string 106)) includes a proximal end section 126 and a distal end section 128, which in turn is terminated by a distal end 130. Distal end 130 of string 106 may be detachably coupled with inner surface 112 of distal port section 122 (as shown in FIG. 1A), although this is not generally required. Proximal end section 126 is defined as the part of string 106 exterior to outer flexible elongated tube layer 102, in an initial operative state of multi-layer endotracheal tube apparatus 100 (i.e., in a manner depicted in FIG. 1A). Distal end section 128 of string 106 is wound substantially around the circumference of distal port section 122 to form a knot 132. The remaining portion of string 106 (i.e., the segment extending from knot 132 to proximal end section 126) is wound substantially helically around outer surface 120, and substantially along the length of collapsible flexible elongated inner tube layer 104. Alternatively, string 106 extends substantially along and within outer flexible elongated tube layer 102 and substantially exteriorly to collapsible flexible elongated inner tube layer 104, without being wound around outer surface 120 of collapsible flexible elongated inner tube layer 104. Multi-layer endotracheal tube apparatus 100 typically further includes an inflatable cuff (not shown), coupled around the exterior surface of outer flexible elongated tube layer 102, substantially in close proximity to distal end 110. Multi-layer endotracheal tube apparatus 100, according to this embodiment of the disclosed technique, may further employ a withdrawal string 134, having one end coupled with proximal end 114 and another end coupled with proximal end section 126, where they come together to form a pulling string 136, as illustrated in FIG. 1A. Alternatively, withdrawal string 134 and pulling string 136 are separate. Withdrawal string 134 (i.e., illustrated in FIG. 1A as being in a loose state) possesses a retraction length, Lᵣ which is substantially greater in length than proximal end section 126. Multi-layer endotracheal tube apparatus 100, including inner surface 118 thereof, is kept sterile prior to intubation.

During intubation, distal end 110 is inserted along with the inflatable cuff (i.e., being at a deflated state) via an airway (not shown) into the trachea (not shown) of a patient (not shown). Multi-layer endotracheal tube apparatus 100 is sufficiently flexible (i.e., the layers thereof) to follow the internal contour of the airway of the patient and concurrently rigid to be advanced into a desired placement location (not shown) within the trachea without crumpling. When distal end 110 reaches the desired placement location, the inflatable cuff is inflated so as to form a seal against the wall of the trachea. Once multi-layer endotracheal tube apparatus 100 is at the desired placement location and the inflatable cuff inflated, ventilation is performed from proximal end 108. As soon as ventilation commences and substantially thereafter, the sterile inner surface 118 becomes exposed to ventilation gases (e.g., air, oxygen) as well as, typically, to microorganisms, secretions, contaminated air, and the like. If collapsible flexible elongated inner tube layer 104 is not removed, bacteria and fungi eventually adhere onto inner surface 118, which becomes a ground for microorganism colonization, as well as a substantially favorable environment for biofilm formation. Furthermore, there is a build-up of secretions on inner surface 118, which over time accumulate and progressively decrease the internal volume (i.e., Vᵢ) available for respiration. In order to prevent or to at least reduce the risk of acquiring or developing medical conditions (e.g., VAP) associated with endotracheal intubation and tracheostromy, a medical practitioner (not shown), at any desired time, may remove collapsible flexible elongated inner tube layer 104 along with material (e.g., secretions) substantially contained within the internal volume thereof, from outer flexible elongated tube layer 102. The removal of collapsible flexible elongated inner tube layer 104 from within outer flexible elongated tube layer 102 will now be described.

According to the disclosed technique, the closure mechanism (i.e., string 106) is operative to close distal port section 122, so as to create a seal, which acts to contain biological material (e.g., secretions, pathogens, microorganisms—not shown) within the internal volume of collapsible flexible elongated inner tube layer 104, especially during the removal of collapsible flexible elongated inner tube layer 104 from outer flexible elongated tube layer 102. This seal ensures that substantially no biological material leaks out from collapsible flexible elongated inner tube layer 104, while collapsible flexible elongated inner tube layer 104 is removed, so as to contaminate remaining exterior layers (i.e., outer flexible elongated tube layer 102).

With reference to FIG. 1B, when pulling string 136 is pulled, string 106 tightens around collapsible flexible elongated tube layer 104 thereby reducing the internal volume thereof. When the internal volume of collapsible flexible elongated tube layer 104 decreases substantially (FIG. 1B) in comparison with that of its initial state (FIG. 1A), collapsible flexible elongated tube layer 104 is said to be in a collapsed state or partially collapsed. The reduction of the internal volume of collapsible flexible elongated tube layer 104 lowers the area of contact with inner surface 112, thus substantially reducing friction when collapsible flexible elongated tube layer 104 is removed from outer flexible elongated inner tube layer 102. As the tension in string 106, between distal end 130 and proximal end 126 increases, knot 132 further tightens around, thus constricting distal end 116 until distal port section 122 substantially closes. After distal port section 122 closes, additional tension applied to string 106 from the pulling thereof, detaches distal end 130 from inner surface 112. When the length of proximal end 126 reaches a length substantially equal to the retraction length, withdrawal string 134 ceases to be in a slack state and tightens. When pulling string 136 is pulled further, there is a substantial increase in tension in string 134 (i.e., also in string 106), which brings about proximal end 114 to emerge from proximal end 108 of outer flexible elongated tube layer 102. The initial infinitesimal state of this emergence is shown in FIG. 1B. When pulling string 136 is pulled even further, rim 124 detaches from inner surface 112 of outer flexible elongated tube layer 102. At this stage, the detachment of rim 124 from inner surface 112 (FIG. 1C), allows withdrawal of collapsible flexible elongated inner tube layer 104 from proximal end 108 of outer flexible elongated tube layer 102. Multi-layer endotracheal tube apparatus 100 enables inner layers (e.g., collapsible flexible elongated inner tube layer 104) to be removed from outer flexible elongated tube layer 102, which possesses kinks and bends (not shown) along the length thereof. It is noted that the removal of inner layers from multi-layer endotracheal tube apparatus 100 may be performed substantially during all the phases of the ventilatory cycle of the patient. It is further noted that the closure mechanism is not limited to a particular implementation and may employ various other methods, such as electromechanical methods (e.g., those which employ miniature electrical motors), magnetic methods, hydraulic methods, and the like.

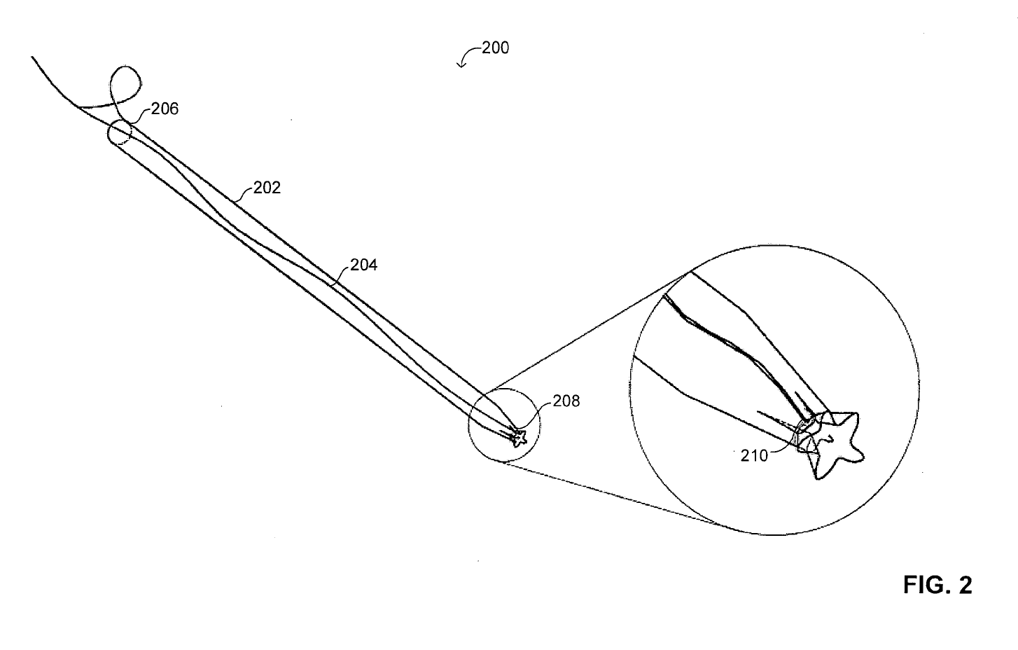

In an alternative embodiment to the disclosed technique, multi-layer endotracheal tube apparatus 100 may employ an inner tube layer, which is party rigid (i.e., not collapsible) along a portion thereof and collapsible along other portions. To illustrate this, reference is now made to FIG. 2, which is a schematic illustration of a partially collapsible flexible elongated inner tube layer, constructed and operative in accordance with an alternative embodiment to the disclosed technique of FIGS. 1A, 1B, and 1C. FIG. 2 illustrates a partially collapsible elongated inner tube layer 202, and a pulling wire 204. Partially collapsible elongated inner tube layer 202 includes a proximal end 206 and a distal end 208. FIG. 2 further illustrates an enlarged view of distal end 208. An outer tube layer is not shown, for as it is substantially similar to outer flexible elongated tube layer 102 of FIGS. 1A, 1B and 1C. According to this embodiment, at least one portion of partially collapsible elongated inner tube layer 202 is collapsible, that of distal end 208 and at least one other portion is rigid (i.e., the portion of the tube excluding distal end 208). Pulling wire 204 is coupled to one end around a circumference of distal end 208 as to form a knot 210. Knot 210 is operative to tighten around the point at which it is disposed, as to constrict distal end 206 at that point. Furthermore, according to this embodiment, pulling wire 204 extends exteriorly along and without being wound around partially collapsible flexible elongated inner tube layer 202. When pulling wire 204 is pulled, knot 210 tightens around distal end 208, which is collapsible, so as to substantially seal distal end 208 and to enable withdrawal (not shown) of partially collapsible flexible elongated inner tube layer 202 from within the outer tube layer.

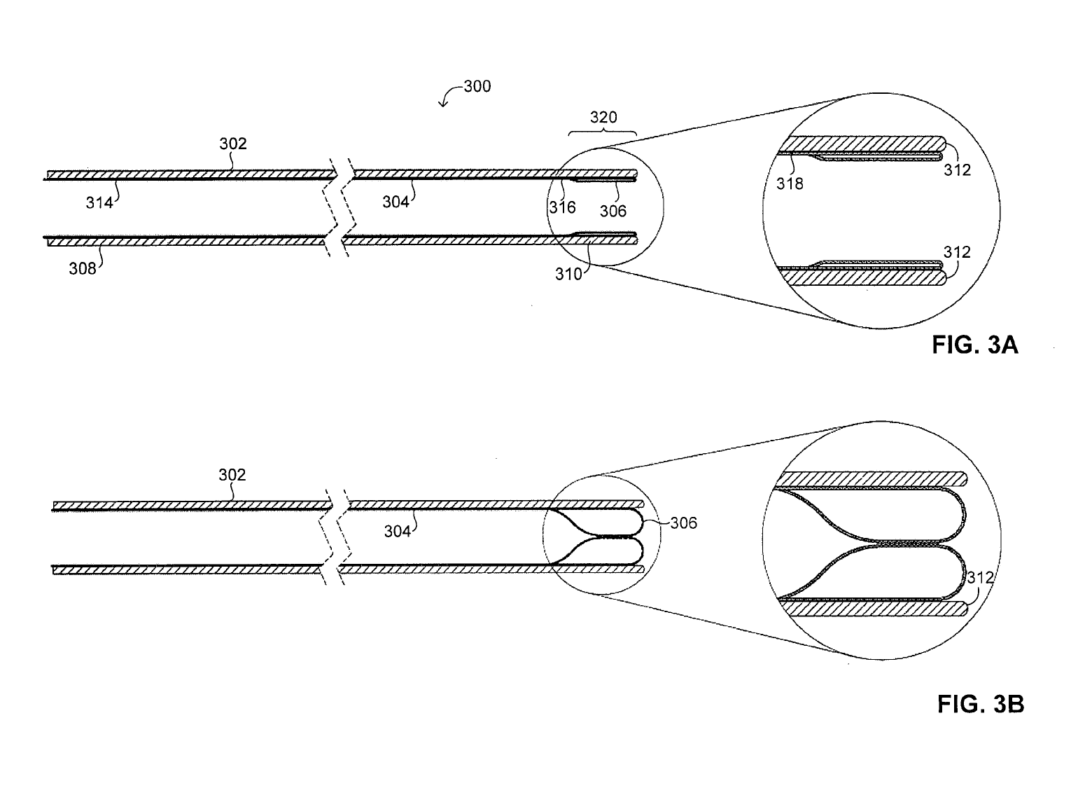

In another alternative embodiment to the disclosed technique of FIGS. 1A, 1B, 1C and 1D, the multi-layer endotracheal tube apparatus employs a closure mechanism that is inflatable. Reference is now made to FIGS. 3A and 3B. FIG. 3A is a schematic illustration of a multi-layer endotracheal tube apparatus, generally referenced 300, utilizing an inflatable closure mechanism, constructed and operative in accordance with another alternative embodiment to the disclosed technique of FIGS. 1A, 1B, and 1C. FIG. 3B is a schematic illustration depicting the multi-layer endotracheal tube apparatus of FIG. 3A in a particular operative state. Multi-layer endotracheal tube apparatus 300 is substantially similar to multi-layer endotracheal tube apparatus 100 of FIGS. 1A, 1B and 1C, except for the inclusion of an inflatable closure mechanism (i.e., an inflatable body) and the exclusion of the string-implemented closure mechanism. Multi-layer endotracheal tube apparatus 300 includes an outer flexible elongated tube layer 302, a plurality (not shown) of inner tube layers, where at least one of these layers is shown as flexible elongated inner tube layer 304, and an inflatable balloon 306. Outer flexible elongated tube layer 302 has a proximal end 308 and distal end 310 that defines an inner surface 312 therebetween. Flexible elongated inner tube layer 304 has a proximal end 314 and a distal end 316, defining an inner surface 318 therebetween. Distal end 316 defines a distal port section 320, a passage through which the outflow and inflow of fluids may be regulated. Inflatable balloon 306 is coupled with distal end 316 of flexible elongated inner tube layer 304 and to distal end 310 of inner surface 312 of outer flexible elongated tube layer 302. FIG. 3A illustrates inflatable balloon 306 in a deflated state. In the inflated state (FIG. 3B), inflatable balloon 306 is inflated via a generally slender tube (not shown), such that distal port section 320 closes, thus preventing the outflow of fluids from within flexible elongated inner tube layer 304 via distal end 316 thereof. It is noted that in an alternative embodiment, inflatable balloon 306 may be a part of flexible elongated inner tube layer 304. According to this alternative, flexible elongated inner tube layer 304 may incorporate a closed (inflatable) volume (not shown), which may be inflated.

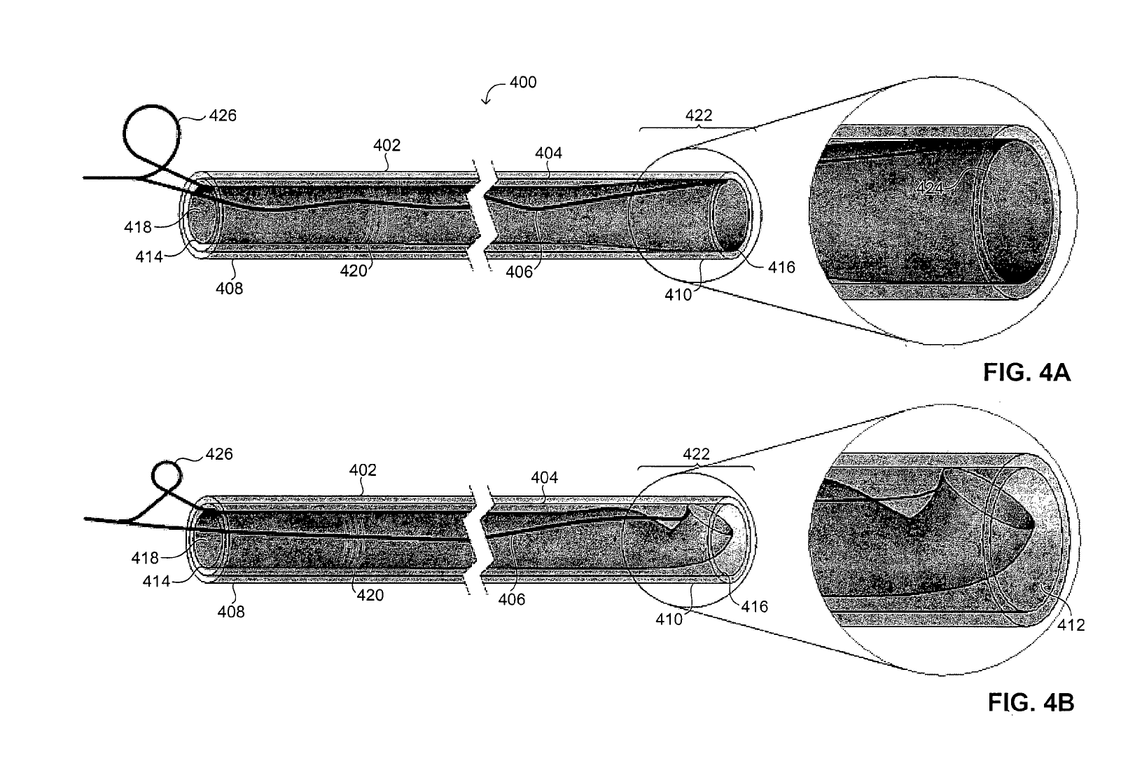

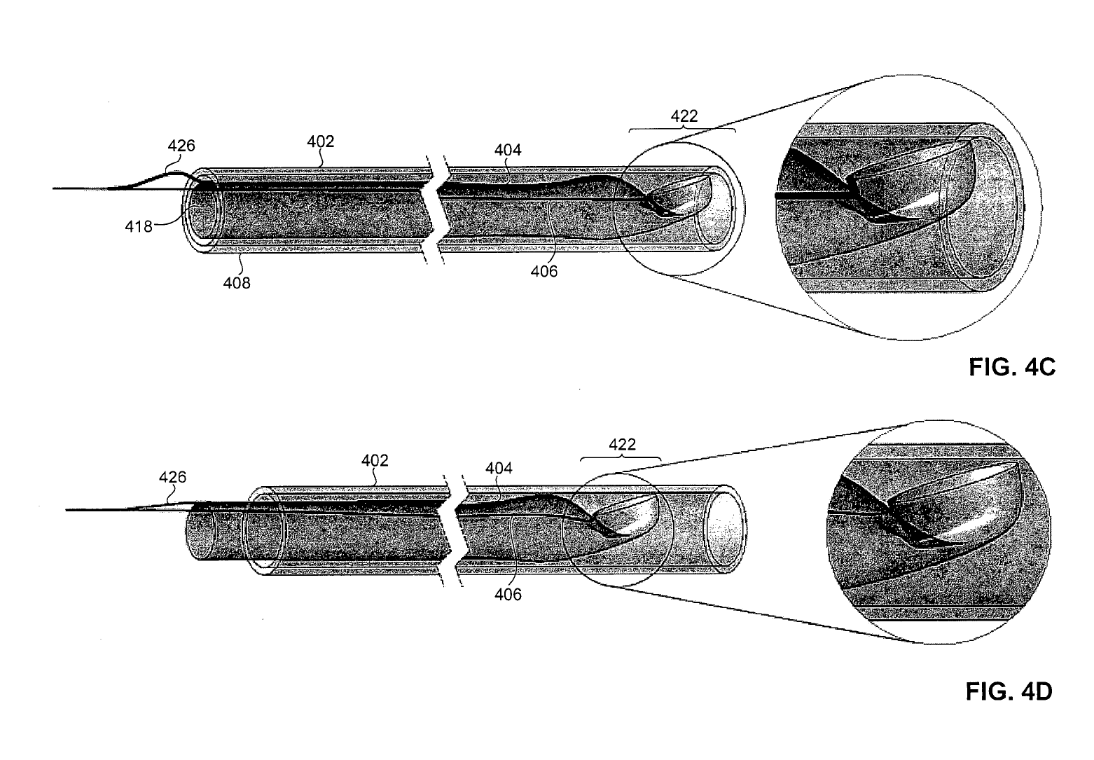

According to another alternative embodiment to the disclosed technique illustrated in FIGS. 1A, 1B and 1C, the distal port section of the inner tube layer is constructed to crumple in a manner that seals the distal port section. To further elaborate, reference is now made to FIGS. 4A, 4B, 4C and 4D. FIG. 4A is a schematic illustration of a multi-layer endotracheal tube apparatus, generally referenced 400, having an inner tube layer that is constructed to at least partially crumple, constructed and operative in accordance with another alternative embodiment of the disclosed technique. FIG. 4B is a schematic illustration of the multi-layer endotracheal tube apparatus of FIG. 4A in a particular operative state. FIG. 4C is a schematic illustration of the multi-layer endotracheal tube apparatus of FIG. 4A in another operative state. FIG. 4D is a schematic illustration of the multi-layer endotracheal tube apparatus of FIG. 3A in a further operative state.

Multi-layer endotracheal tube apparatus 400 (FIG. 4A), substantially similar to multi-layer endotracheal tube apparatus 100, illustrated in FIGS. 1A, 1B and 1C, includes an outer flexible elongated tube layer 402 (similar to outer flexible elongated tube layer 102), a plurality (not shown) of inner tube layers, where at least one of these layers is shown as flexible elongated inner tube layer 404, and a withdrawal string 406. Outer flexible elongated tube layer 402 has a proximal end 408 and a distal end 410, both of which define an inner surface 412 therebetween (more distinctly shown in FIG. 4B). Flexible elongated inner tube layer 404 has a proximal end 414 and a distal end 416, which defines an inner surface 418 and an outer surface 420 therebetween. Distal end 416 defines a distal port section 422, which in turn is terminated by rim 424. Flexible elongated inner tube layer 404 extends substantially within, and along the inner length of outer flexible tube layer 402. Rim 424 of distal end 416 is detachably coupled along an inner closed circumference of inner surface 412. Withdrawal string 406 extends substantially along the length of elongated inner tube layer 404. Withdrawal string 406 generally has two ends, one of which is coupled to distal end 410 of elongated inner tube layer 404, the other end exits from proximal end 408 of outer flexible elongated tube layer 402 and is coupled with one end of a pulling string 426. The other end of pulling string 426 is coupled to proximal end 414 of flexible elongated inner tube layer 404.

When withdrawal string is pulled, rim 424 detaches from inner surface 412 (FIG. 4B). In this present alternative embodiment to the disclosed technique, distal port section 422 is operative to crumple (i.e., wrinkle, undergo structural collapse), when withdrawal string 406 is sufficiently pulled. FIG. 4B illustrates a preliminary state in the crumpling process of distal end section 408. In FIG. 4C, distal port section 422 is shown to be further crumpled, such that distal port section 422 is sealed. This seal functions as containment for keeping material (i.e., fluid or solid), held within the internal volume of flexible elongated inner tube layer 404, from escaping into a subsequent inner tube layer (not shown) or into outer flexible elongated tube layer 402. When distal port section 422 substantially seals, the process of withdrawing flexible elongated inner tube layer 404 from within outer flexible elongated tube layer 402 (FIG. 4D) may commence. FIG. 4D shows a particular instant in the withdrawal process. Pulling string 426 tightens thus facilitating the withdrawal of flexible elongate inner tube layer 404 from outer flexible elongated tube layer 402 via proximal end 408.

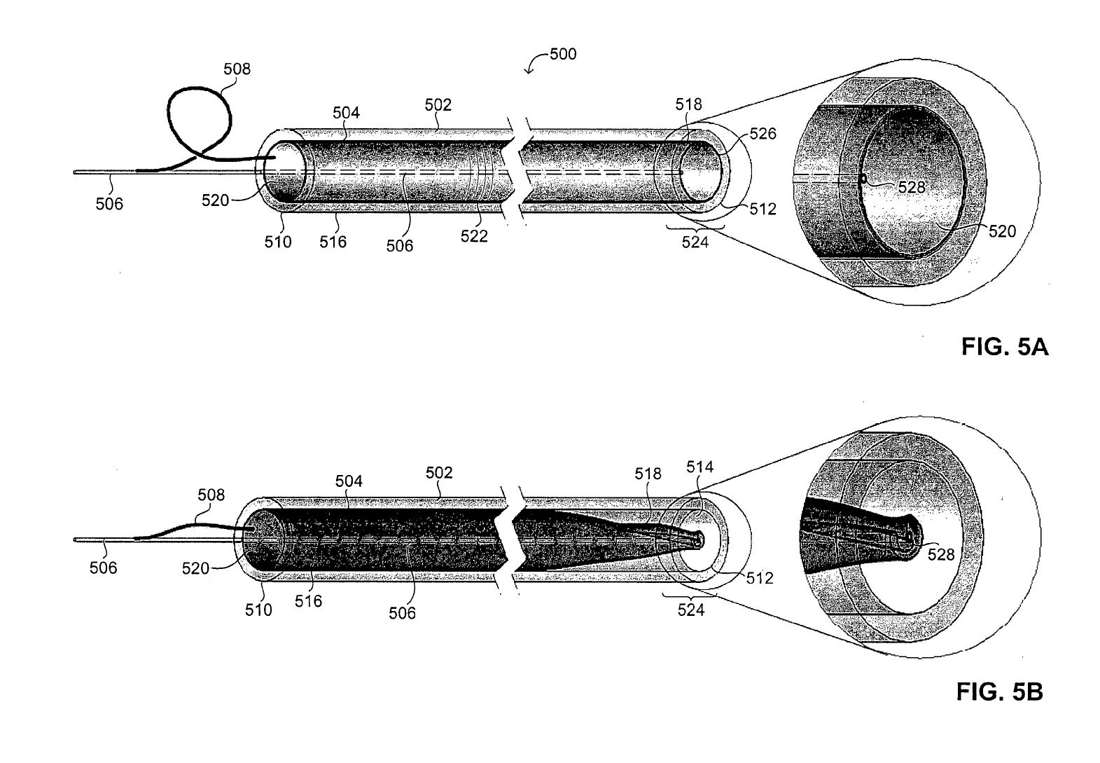

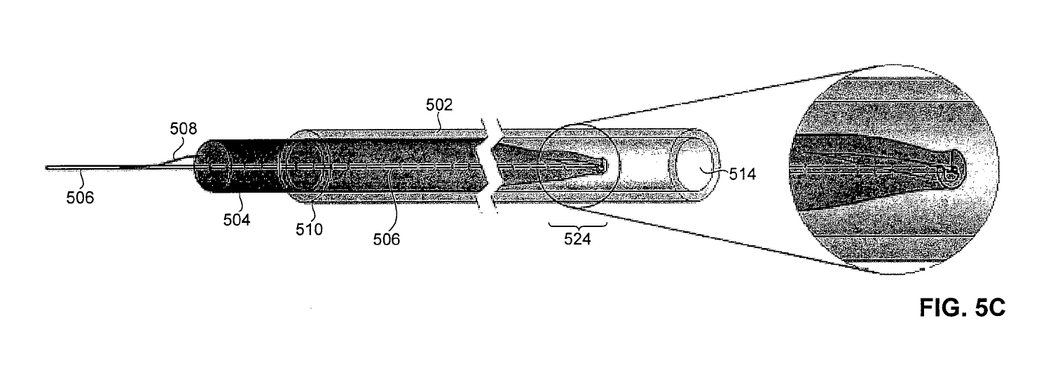

According to a further alternative embodiment to the disclosed technique of FIGS. 1A, 1B and 1C, the distal port section of the inner tube layer is operative to be rolled close by employing a flexible elongated rod. To elaborate further, reference is now made to FIGS. 5A, 5B, and 5C. Stretch FIG. 5A is a schematic illustration of a multi-layer endotracheal tube apparatus, generally referenced 500, having an inner tube layer that is operative to roll close, constructed and operative in accordance with a further alternative embodiment of the disclosed technique. FIG. 5B is a schematic illustration of the multi-layer endotracheal tube apparatus of FIG. 5A in a particular operative state. FIG. 5C is a schematic illustration of the multi-layer endotracheal tube apparatus of FIG. 5A in another operative state. Multi-layer endotracheal tube apparatus 500 is substantially similar to multi-layer endotracheal tube apparatus 100 of FIGS. 1A, 1B, and 1C. Multi-layer endotracheal tube apparatus 500 includes an outer flexible elongated tube layer 502, a plurality (not shown) of inner tube layers, where at least one of these layers is shown as flexible elongated inner tube layer 504, and a flexible elongated rod 506 (e.g., a torque wire, and the like). Multi-layer endotracheal tube apparatus 500 may further include a pulling string 508.

Outer flexible elongated tube layer 502 has a proximal end 510 and a distal end 512, both of which define an inner surface 514 therebetween (shown more distinctly in FIG. 5B). Flexible elongated inner tube layer 504 has a proximal end 516 and a distal end 518, which define an inner surface 520 and an outer surface 522 therebetween. Distal end 518 defines a distal port section 524, which in turn is terminated by rim 526. Flexible elongated inner tube layer 504 extends substantially within, and along the inner length of outer flexible elongated tube layer 502 (FIG. 5A). Rim 526 of distal end is detachably coupled along an inner closed circumference of inner surface 514. In general, part of flexible elongated rod 506 is exterior to flexible elongated inner tube layer 504. A substantial part of flexible elongated rod 506 extends within and along the inner length of outer flexible tube layer 502, ending at a terminus 528 (i.e., a rod distal end) that is coupled with inner surface 520 of flexible elongated inner tube layer 504. Alternatively, terminus 528 is coupled with outer surface 522. Pulling string 508 is coupled with flexible elongated inner tube layer 504.

When flexible elongated rod 506 is rotated (FIG. 5B), rotation and torque develops at distal end 518 and rim 526 detaches from inner surface 514. As flexible elongated rod 506 is rotated further, more torque is transmitted progressively along the length of flexible elongated inner tube layer 504 toward proximal end 516 thereof. Flexible elongated inner tube layer 504 constricts progressively, as further rotation causes distal port section 524 to roll close (FIG. 5B). When distal port section 526 is sufficiently closed (i.e., when fluids are substantially prevented from escaping therefrom), flexible elongated inner tube layer 504 may then be withdrawn from within outer flexible elongated tube layer 502, as shown in FIG. 5C.

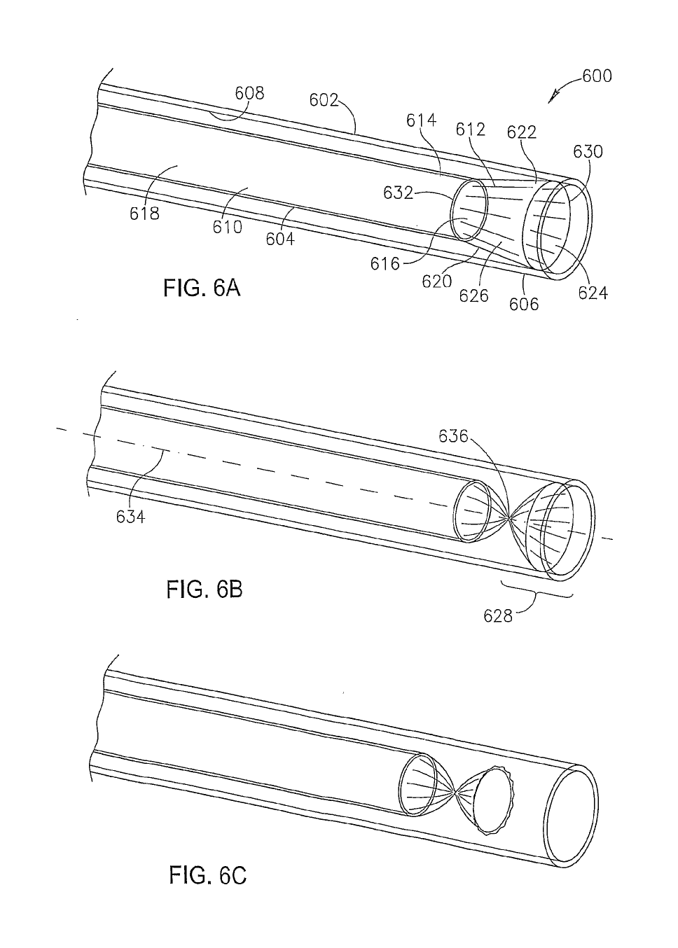

According to another embodiment of the disclosed technique, the multi-layer endotracheal tube apparatus is in part, of hybrid construction, combining two components, one possessing a greater torsional rigidity (or torsional stiffness) than the other. The component having a substantially higher torsional rigidity is embodied in the form of a flexible yet rigid, tubular structure, whereas the component with the substantially lower torsional rigidity is twistable and may be embodied as a film (e.g., a foil, plastic film) possessing high tensile strength. The twistable component functions as the closure mechanism. Alternatively, each component may have different a different elastic modulus than the other. To further elaborate, reference is now made to FIGS. 6A, 6B, and 6C. FIG. 6A is a schematic illustration of a multi-layer endotracheal tube apparatus of hybrid construction, generally referenced 600, constructed and operative according to another embodiment of the disclosed technique. FIG. 6B is a schematic illustration depicting an operative state of the multi-layer endotracheal tube apparatus of FIG. 6A. FIG. 6C is a schematic illustration depicting another operative state of the multi-layer endotracheal tube apparatus of FIG. 6A. Multi-layer endotracheal tube apparatus 600 (FIG. 6A) includes an outer flexible elongated tube layer 602, and at least one (i.e., usually a plurality of) inner tube layer 604. Outer flexible elongated tube layer 602 has a distal end 606 and a proximal end (not shown), both of which define an inner surface 608 therebetween. Inner tube layer 604 is essentially constructed from an elongated flexible rigid tube section 610 and a twistable tube section 612. Elongated flexible rigid tube section 610 possesses a substantially greater torsional rigidity than that of twistable tube section 612. Elongated flexible rigid tube section 610 has a distal end 614 and a proximal end (not shown), both of which define an inner surface 616 and an outer surface 618 therebetween. Twistable tube section 612 has a proximal end 620 and a distal end 622, both which define an inner surface 624 and an outer surface 626 therebetween. Distal end 622 defines a distal port section 628, which in turn is terminated by a distal rim 630. Proximal end 620 is terminated by a proximal rim 632. Elongated flexible rigid tube section 610 extends substantially within, and along the inner length of outer flexible elongated tube layer 602. Distal rim 630 is detachably coupled along an inner closed circumference of inner surface 608 of outer flexible elongated tube layer 602. Proximal rim 632 is coupled along a substantially closed circumference of distal end 614 of elongated flexible rigid tube section 610. The coupling of distal rim 630 with inner surface 608, as shown in FIG. 6A, prevents fluids, entering via distal port section 628, from seeping into the outer surfaces 626 and 618, as well as inner surface 608. In effect, this coupling, in the state illustrated in FIG. 6A, functions as a barrier that shields sterilized surfaces of multi-layer endotracheal tube apparatus 600 from other surfaces, those which have become exposed to secretions, contamination, pathogens, and the like.

In the present embodiment of the disclosed technique, twistable tube section 612 is operative to twist close, so as to seal distal port section 628 and thus, substantially contain biological material within the internal volume of elongated flexible rigid tube section 610. This sealing of distal port section 628 is brought about by rotating elongated flexible rigid tube section 610 at the proximal end thereof, in relation to outer flexible elongated tube layer 602, substantially about an axis 634 (FIG. 6B). Axis 634 is the central axis extending along the length of elongated flexible rigid tube section 610, and duly following the curvature (not shown) thereof. It is noted that the rotation of elongated flexible rigid tube section 610 within outer flexible elongated tube layer 602 may be facilitated by coating outer surface 618 and inner surface 608 with a lubrication layer (not shown), so as to reduce friction between these engaging surfaces during rotation. It is further taken into account that for each point along the length of outer flexible elongated tube layer 602, the inner cross-sectional diameter thereof is substantially less than the greatest outer cross-sectional diameter of elongated flexible rigid tube section 610.

Torque applied to the proximal end of elongated flexible rigid tube section 610, brings about elongated flexible rigid tube section 610 to turn (i.e., rotate) within outer flexible elongated tube layer 602. This applied torque, substantially transmitted to distal end 614, is then exerted on proximal end 620 of twistable tube section 612. As proximal rim 632 rotates (i.e., relative to outer flexible elongated tube layer 602), while distal rim 630 remains stationary, the torsion in twistable tube section 612 increases causing it to wrench (FIG. 6B). The rotation of elongated flexible rigid tube section 610 distorts twistable tube section 614 so that a constriction 636 forms (FIG. 6B) at a location along the length of twistable tube section 614 (i.e., between distal rim 630 and proximal rim 632). Thus twistable tube section 614 is twisted to close at constriction 636, effectively sealing distal port section 628 from both influx and efflux of fluids. Therefore, after closure, any biological material contained within the internal volume of elongated flexible rigid tube section 610 remains retained and enclosed therein. The location along twistable tube section 614 at which constriction 636 develops may be controlled such that it is closer (not shown) to distal rim 630 than to proximal rim 632. Implementation of this may involve, for example, the use of a material possessing nonuniform torsional rigidity along its length (e.g., having a varying thickness, varying torsional coefficients).

Once twistable tube section 614 is twisted to close, further rotation of elongated flexible rigid tube section 610 causes distal rim 630 to detach from being coupled to inner surface 608 of elongated flexible rigid tube section 610 (FIG. 6C). At this stage, inner tube layer 604 is removed from outer flexible elongated tube layer 602 via the proximal end of outer flexible elongated tube layer 602.

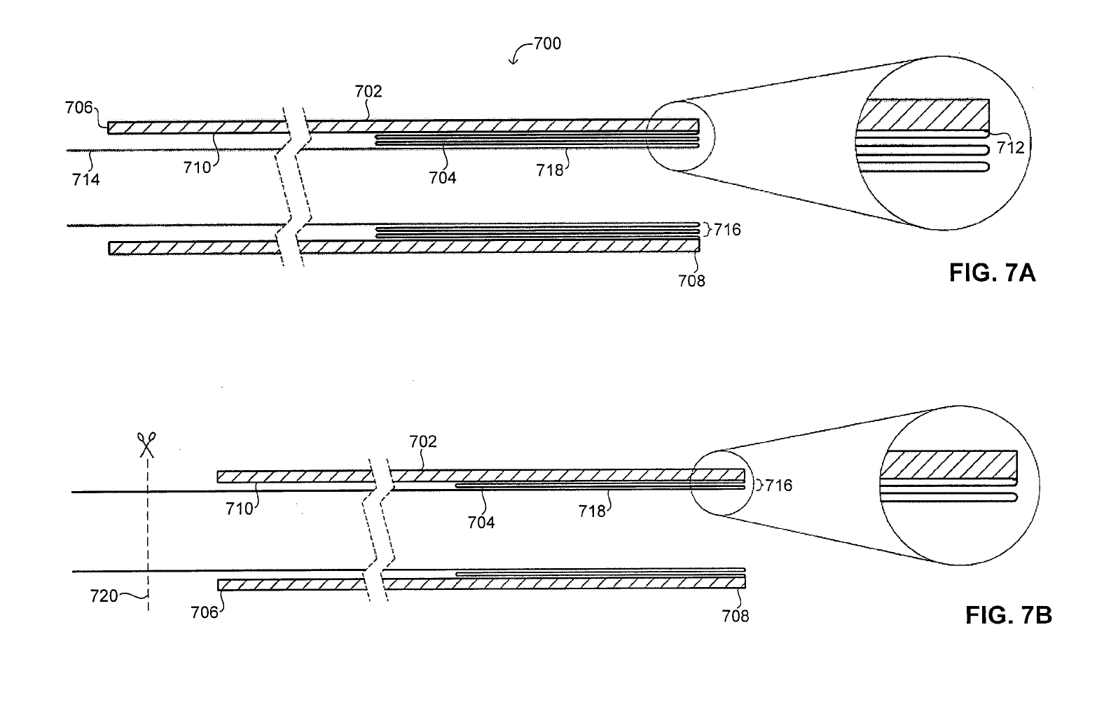

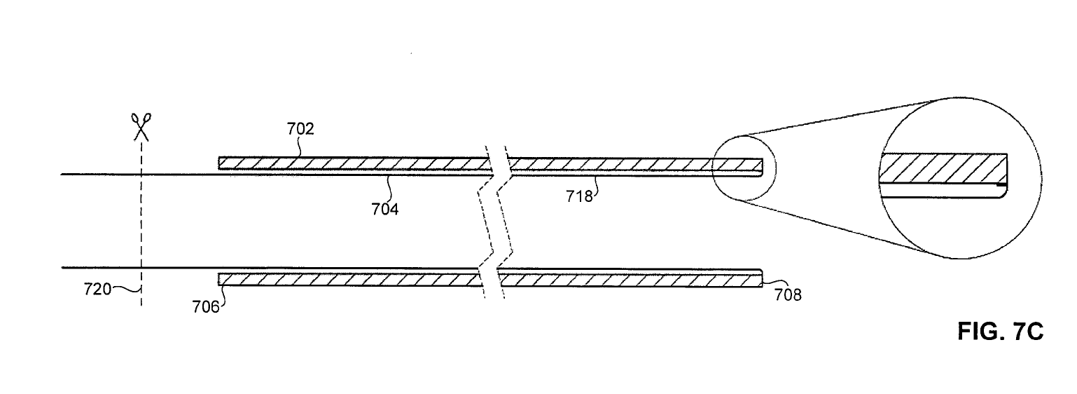

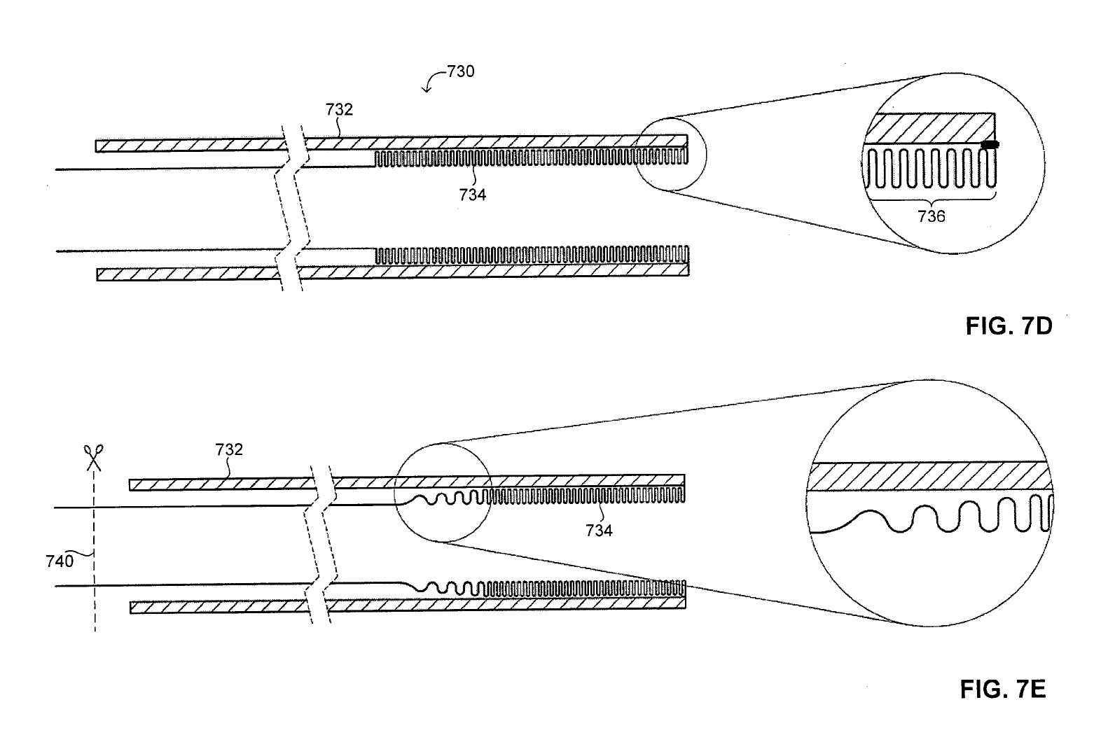

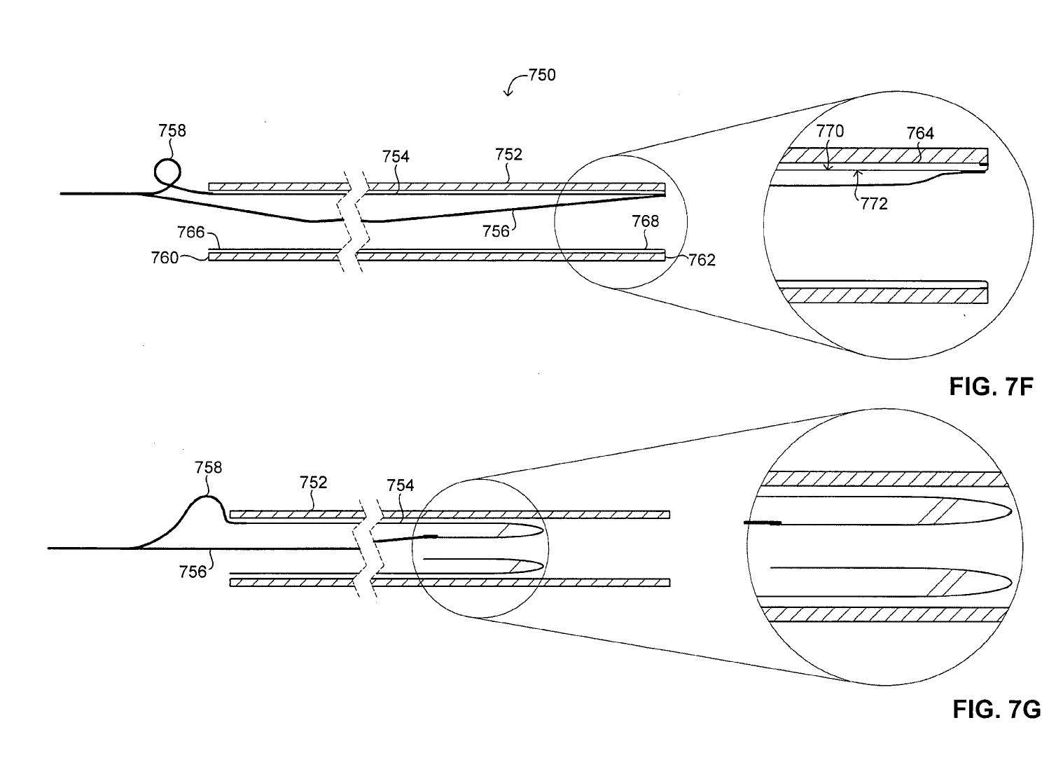

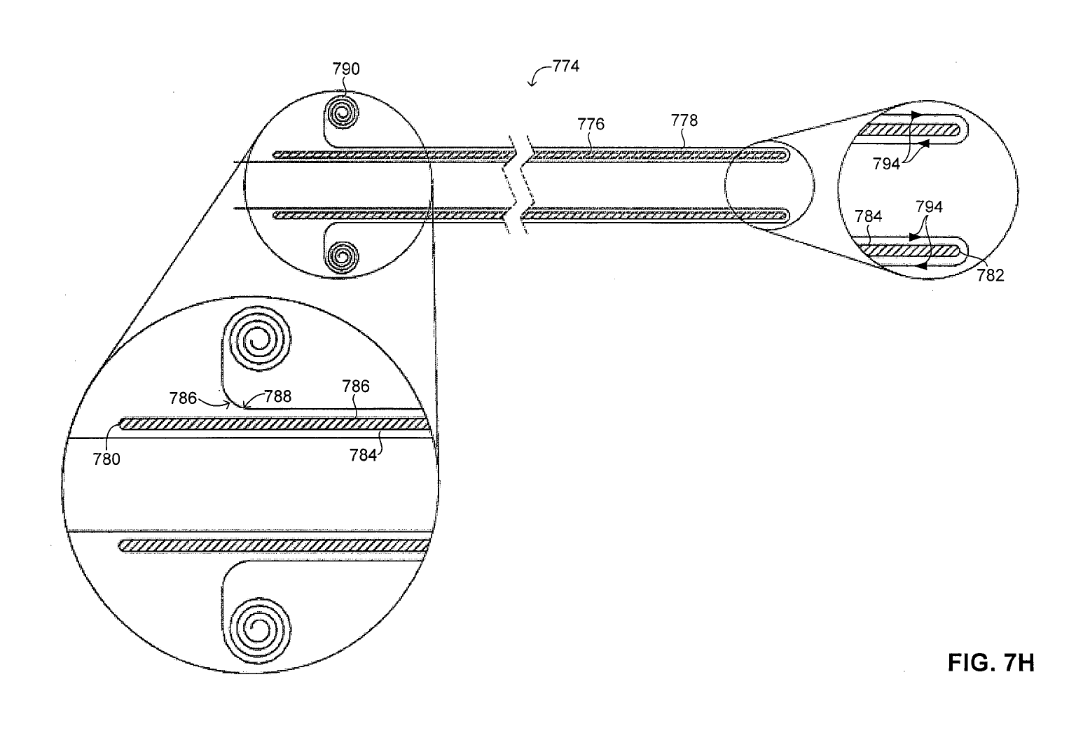

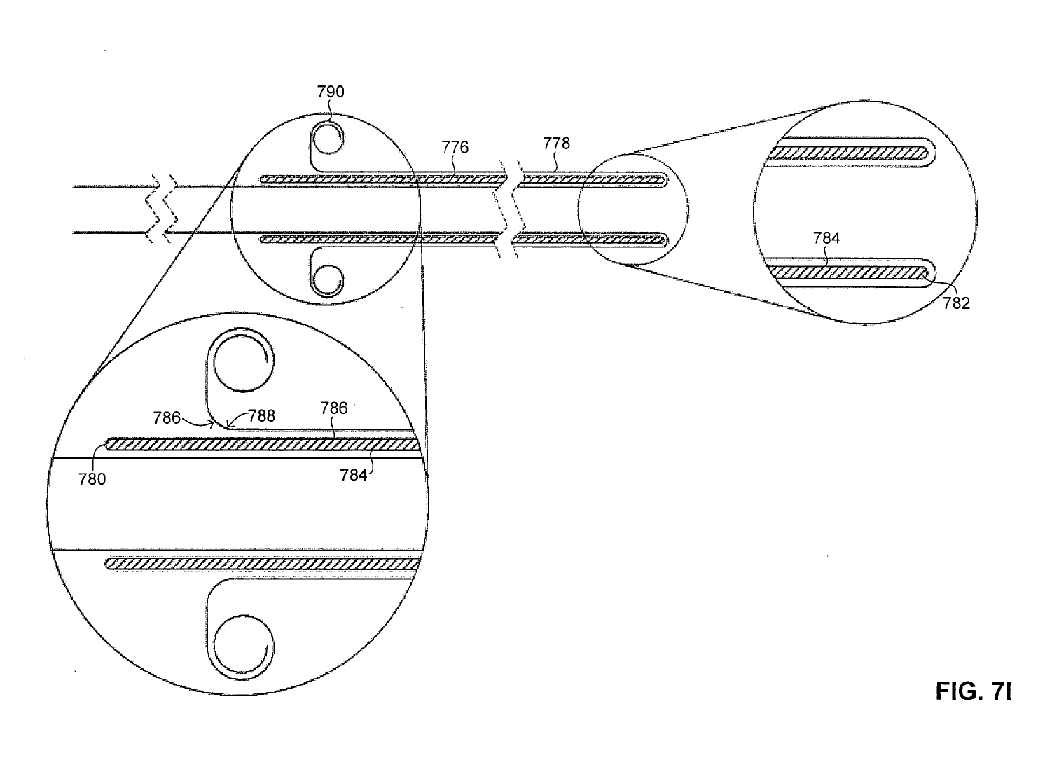

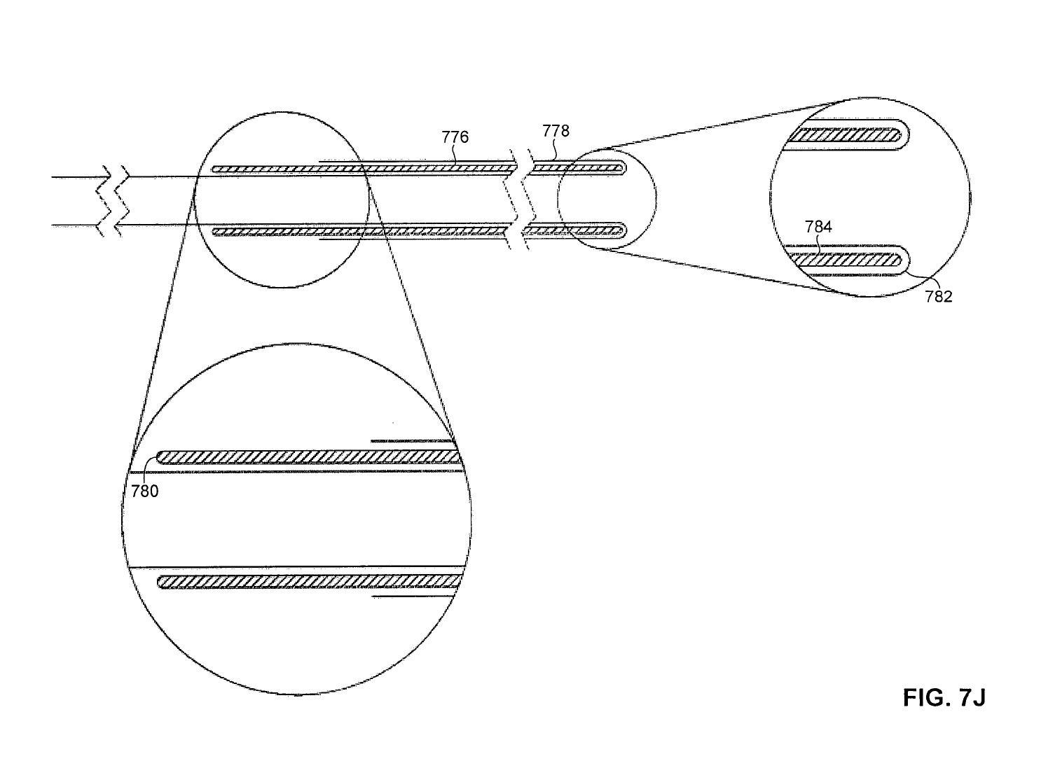

According to a further embodiment of the disclosed technique, the multi-layer endotracheal tube apparatus includes a plurality of inner tube layers, which are layered (i.e., stacked) onto each other, such that each layer is operative to peel (i.e., detach) from its successive layer, as to enable its withdrawal from the outer tube layer. When the most inner tube layer is withdrawn from the outer tube layer, biological material adhered thereto is substantially removed along with that inner tube layer. Reference is now made to FIGS. 7A, 7B and 7C. FIG. 7A is a schematic cross-sectional illustration in longitudinal section view of a multi-layer endotracheal tube apparatus, generally referenced 700, having a parallel inner tube layer stack, constructed and operative in accordance with a further embodiment of the disclosed technique. FIG. 7B is a schematic cross-sectional illustration in longitudinal section view of the multi-layer endotracheal tube apparatus of FIG. 7A in a particular operative state. FIG. 7C is a schematic cross-sectional illustration in longitudinal section view of the multi-layer endotracheal tube apparatus of FIG. 7A in another operative state.

Multi-layer endotracheal tube apparatus 700 (FIG. 7A) includes an outer flexible elongated tube layer 702, and a flexible elongated parallel inner layer stack 704. Outer flexible elongate tube layer 702 is substantially tubular and has a proximal end 706 and a distal end 708, which define an inner surface 710 therebetween. Flexible elongated parallel inner layer stack 704 has a head terminus 712 and a tail terminus 714. Flexible elongated parallel inner stack extends substantially parallel along the inner length of outer flexible elongated tube layer 702 in a parallel serpentine comportment, as to form a plurality of parallel layers 716 (FIG. 7A). The inner most layer of layers 716 is defined as the layer contiguous (i.e., having the closest distance) to outer flexible elongated tube layer 702, while the outer most layer of layers 716 is defined as being the relatively farthest. The outer most layer of layers 716 defines an exposed surface 718, substantially exposed to biological material. Head terminus 712 is detachably coupled to inner surface 710 at distal end 708 and tail terminus 714 terminates substantially exterior to outer flexible elongated tube layer 702. The initial state of multi-layer endotracheal tube apparatus 700 is shown in FIG. 7A. Although for the purposes of elucidating the disclosed technique, successive formed layers are shown not be in contact, it is nonetheless noted that successive layers may substantially be in contact.

Over time, following insertion of multi-layer endotracheal tube apparatus 700 into the patient, exposed surface 718 of the outer most layer of layers 716, becomes exposed to biological material. For removing the outer most layer, tail terminus 714 is pulled a certain distance (e.g., the distance equaling the length of outer flexible elongated tube layer 702) from proximal end 706 (FIG. 7B), after which it is cut 720 and disposed of (not shown). It is noted that successive inner layers of layers 716 do not make contact with exposed layer 718 during the removal of the exposed layer 718. When the inner most layer becomes the outer most layer (i.e., remains the last layer) (FIG. 7C), it may be removed (not shown), whereby head terminus 712 detaches (not shown) from inner surface 710 of outer flexible elongated tube layer 702.

Reference is now made to FIGS. 7D and 7E. FIG. 7D is a schematic cross-sectional illustration in longitudinal section view of a multi-layer endotracheal tube apparatus, generally referenced 730, having a perpendicular inner tube layer stack, constructed and operative in accordance with another embodiment of the disclosed technique. FIG. 7E is a schematic cross-sectional illustration in longitudinal section view of the multi-layer endotracheal tube apparatus of FIG. 7D in a particular operative state. Multi-layer endotracheal tube apparatus 730 (FIG. 7D) is substantially similar to multi-layer endotracheal tube apparatus of FIGS. 7A, 7B and 7C, although having a perpendicular inner layer stack. Multi-layer endotracheal tube apparatus 730 includes an outer flexible elongated tube layer 732, and a flexible elongated perpendicular inner layer stack 734. Flexible elongated perpendicular inner stack 734 extends along the inner length of outer flexible elongated tube layer 732 in a perpendicular serpentine comportment (i.e., perpendicular to the length of outer flexible elongated tube layer 732), as to form a plurality of parallel layers 736 (FIG. 7A). The initial state of multi-layer endotracheal tube apparatus 730 is shown in FIG. 7D. Although for the purposes of elucidating the disclosed technique, successive formed layers are shown not be in contact (see enlargement in FIG. 7D), it is nonetheless noted that successive layers may substantially be in contact. The removal of layers 736 (FIG. 7E) necessitates pulling flexible elongated perpendicular inner stack 734, after which it may be cut 740 and disposed of (not shown). Alternatively, the flexible elongated inner layer stack may be disposed at various angles (e.g., 45°, 60°) in relation to the outer tube layer (not shown).

Reference is now made to FIGS. 7F and 7G. FIG. 7F is a schematic cross-sectional illustration in longitudinal section view of a multi-layer endotracheal tube apparatus, generally referenced 750, having inner tube layers that peel from within the outer tube layer, constructed and operative in accordance with a further embodiment of the disclosed technique. FIG. 7G is a schematic cross-sectional illustration in longitudinal section view of the multi-layer endotracheal tube apparatus of FIG. 7F in a particular operative state.

Multi-layer endotracheal tube apparatus 750 (FIG. 7F) includes an outer flexible elongated tube layer 752, a plurality (not shown) of inner tube layers, where at least one of these layers is shown as flexible elongated inner tube layer 754, a withdrawal string 756, and may optionally include a pulling string 758. Outer flexible elongated tube layer 752 has a proximal end 760 and a distal end 762, both of which define an inner surface 764. Flexible elongated inner tube layer 754 has a proximal end 766 and a distal end 768, which define an inner surface 770 and an outer surface 772. Distal end 768 is detachably coupled along an inner closed circumference of inner surface 764 at distal end 762. Flexible elongated inner tube layer 754 extends substantially within, and along the inner length of outer flexible tube layer 752. Withdrawal string 756 extends substantially along the length of elongated inner tube layer 752. Withdrawal string 756 generally has two ends, one of which is coupled to distal end 768 of elongated inner tube layer 754, the other end exits from proximal end 760 of outer flexible elongated tube layer 752 and is coupled with one end of pulling string 758. Alternatively, withdrawal string 756 is not coupled with pulling string 758. The other end of pulling string 758 is coupled to proximal end 766 of flexible elongated inner tube layer 754.

To remove flexible elongated inner tube layer 754, withdrawal string 756 is pulled from proximal end 760, thereby causing distal end 768 to detach from inner surface 764 of distal end 762. As withdrawal string 756 is pulled, flexible elongated inner tube layer 754 peels away from outer flexible elongated tube layer 752, thereby folding upon itself (i.e., distal end 768 fold upon the rest (remaining portion) of flexible elongated inner tube layer 754, as shown in FIG. 7G). As flexible elongated inner tube layer 754 progressively detaches (i.e., peels) from outer flexible elongated tube layer 752, inner surface 764 of outer flexible inner tube layer does not come into contact with inner surface 770 (i.e., the surface exposed to biological material, shown in the initial operative state in FIG. 7F). Pulling string 758 may additionally be employed to facilitate removal of flexible elongated inner tube layer 754 from within outer flexible elongated tube layer 752.

Reference is now made to FIGS. 7H, 7I and 7J. FIG. 7H is a schematic cross-sectional illustration in longitudinal section view of a multi-layer endotracheal tube apparatus, generally referenced 774, employing external-body inner layer dispensation, constructed and operative in accordance with another embodiment of the disclosed technique. FIG. 7I is a schematic cross-sectional illustration in longitudinal section view of the multi-layer endotracheal tube apparatus of FIG. 7H in a particular operative state. FIG. 7J is a schematic cross-sectional illustration in longitudinal section view of the multi-layer endotracheal tube apparatus of FIG. 7H in another operative state.

Multi-layer endotracheal tube apparatus 774 (FIG. 7H) includes an outer flexible elongated tube layer 776, and a flexible elongated layer 778. Outer flexible elongated tube layer 776 is substantially tubular and has a proximal end 780 and a distal end 782, which define an inner surface 784 and an outer surface 786. Flexible elongated layer 778 has two surfaces (i.e., front and back), which are denoted as an engaging surface 786 and an exposure surface 788. In the initial state, illustrated in FIG. 7H, part of flexible elongated layer 776 is curled into a circular stack 790, located externally to the patient. The part of flexible elongated layer 778 that remains, extends along outer surface 786, up to where it turns at distal end 782 (see arrows 794 in top enlargement), to follow the curvature of outer flexible elongated tube layer 776 along inner surface thereof, and terminating in the periphery of proximal end 780. Following insertion of multi-layer endotracheal tube apparatus 774 into the patient, exposure surface 788 becomes exposed to biological material. Both the biological material and the exposed exposure surface 788 may be removed by pulling flexible elongated layer 778 from proximal end 780 (FIG. 7I). It is noted that there is substantial isolation, with respect to biological material exposure between engaging surface 786 (i.e., which contacts inner and outer surfaces 784 and 786) and exposure surface 788. Over time, as more of flexible elongated layer 778 is removed, more of circular stack 790 is used until it is completely expended (FIG. 7J).

According to another embodiment of the disclosed technique, the multi-layer endotracheal tube apparatus incorporates a laryngeal mask portion, at least one flexible detachable film layer, and a flexible detachable film layer closure mechanism. The laryngeal mask portion is coupled with an outer flexible elongated tube layer. The laryngeal mask includes an inflatable laryngeal cuff having an outer periphery and an inner periphery. The flexible detachable film layer detachably coats the inner periphery of the inflatable laryngeal cuff along a peripheral edge thereof. The detachable film layer closure mechanism is operative to close the flexible detachable film layer so as to form a closed volume and to further enable the withdrawal of this closed volume, formed from the flexible detachable film layer, from the outer flexible elongated tube layer.

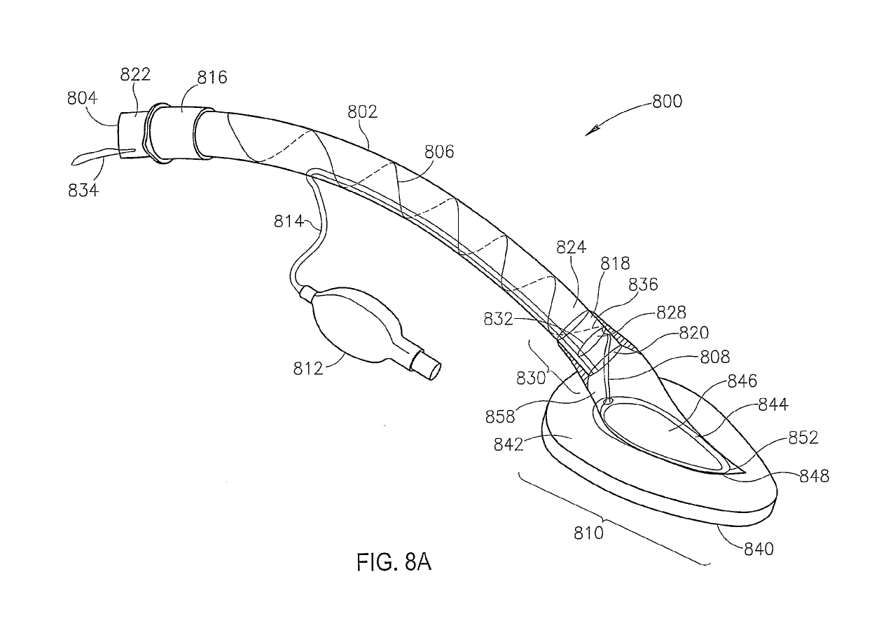

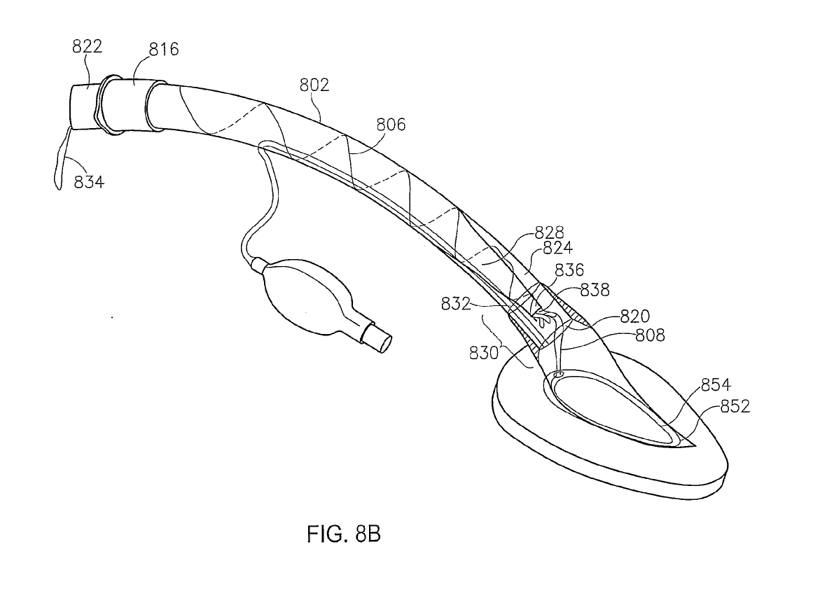

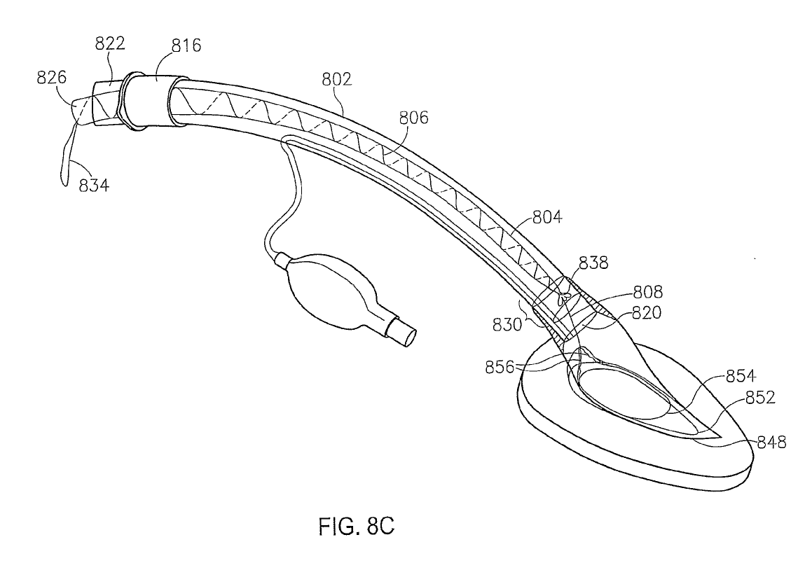

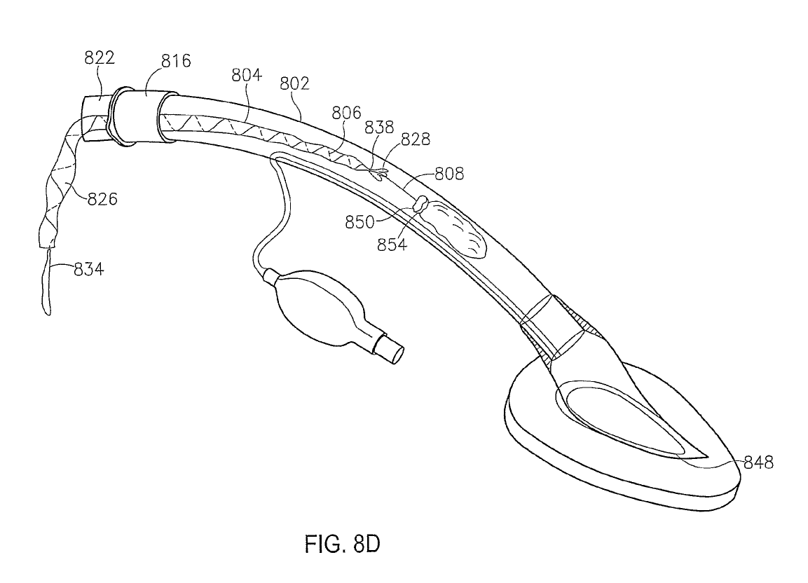

More particularly, reference is now made to FIGS. 8A, 8B, 8C and 8D. FIG. 8A is a schematic illustration of a multi-layer endotracheal tube apparatus including a laryngeal mask portion, generally referenced 800, constructed and operative in accordance a further embodiment of the disclosed technique. FIG. 8B is a schematic illustration of the multi-layer endotracheal tube apparatus including the laryngeal mask portion of FIG. 8A, in a particular operative state. FIG. 8C is a schematic illustration of the multi-layer endotracheal tube apparatus including the laryngeal mask portion of FIG. 8A, in another operative state. FIG. 8D is a schematic illustration of the multi-layer endotracheal tube apparatus including the laryngeal mask portion of FIG. 8A, in a further operative state. With reference to FIG. 8A, multi-layer endotracheal tube apparatus 800 includes an outer flexible elongated tube layer 802, at least one flexible elongated inner tube layer 804, an endotracheal tube string 806, a drawstring 808, and a laryngeal mask portion 810. Multi-layer endotracheal tube apparatus 800 may further include an inflation device 812 for inflating the inflatable part of laryngeal mask portion 810 via an inflation tube 814. Outer flexible elongated tube layer 802 has a proximal end 816 and a distal end 818 defining an inner surface 820 therebetween. Flexible elongated inner tube layer 804 has a proximal end 822 and a distal end 824 so as to define an outer surface 826 (shown more distinctly in FIGS. 8C and 8D) and an inner surface 828 therebetween, and a distal port section 830. Flexible elongated inner tube layer 804 extends substantially within and at least partially along inner length of outer flexible elongated tube layer 802. Distal end 824 defines a rim 832 that is detachably coupled along an inner substantially closed circumference of inner surface 820. Endotracheal tube string 806, having a string proximal section 834 and a string distal section 836, extends substantially along length of flexible elongated inner tube layer 804 and further wound substantially helically around outer surface 826. String distal section 836 may be detachably coupled with inner surface 820. Part of distal section 836 winds about distal port section 830 so as to form a constriction 838 (FIGS. 8B, 8C and 8D) when proximal section 834 is substantially pulled.

Laryngeal mask portion 810 includes an inflatable laryngeal cuff 840 having an outer periphery 842 and an inner periphery (i.e., not shown—opposite to outer periphery 842 of inflatable cuff 840) that defines a cavity 844 having an opening 846. Opening 846 is in communication with flexible elongated inner tube layer 804, and further defines a peripheral edge 848. Flexible detachable film layer 850 (more distinctly shown in FIG. 8D) covers and is detachably coupled to the inner periphery, in a manner that substantially follows the circumference of peripheral edge 848.

Flexible detachable film layer 850 has a rim 852, which defines a flexible detachable film inlet 854 (shown more distinctly in FIG. 8C), along which drawstring 808 substantially encircles (i.e., follows the perimeter thereof). Drawstring 808 is substantially circumferentially coupled along the perimeter of rim 852. Flexible detachable film inlet 854 is detachably circumferentially coupled with peripheral edge 848. Drawstring 808 ends 856 (more distinctly shown in FIG. 8C) are coupled with string distal section 836, however, ends 856 may alternatively be coupled with flexible elongated inner tube layer 804.

Laryngeal mask portion 810 may further include a coupling 858 for coupling inflatable laryngeal cuff 840 with outer elongated flexible tube layer 802 (as shown in FIGS. 8A-8D). Alternatively, laryngeal mask portion 810 may be directly coupled with outer elongated flexible tube layer 802 (e.g., through an annular aperture in said inflatable laryngeal mask—not shown).

Multi-layer endotracheal tube apparatus 800 is typically employed for supraglottic airway management (e.g., in various medical emergencies, anesthesia, and the like) such that laryngeal inflatable cuff 840 is inserted (in its deflated state) and positioned into the pharynx (not shown) of a patient. When laryngeal inflatable cuff 840 is inserted by following the natural curvature of the oropharynx (not shown) and positioned at the desired placement location (e.g., the pharynx), it is inflated so that outer periphery 842 substantially forms a seal with the surrounding tissue and further secures laryngeal mask portion 810 in place. The initial operative state of multi-layer endotracheal tube apparatus 800 is shown in FIG. 8A.

According to the disclosed technique, multi-layer endotracheal tube apparatus 800 employs two closure mechanisms (i.e., endotracheal tube string 806 and drawstring 808) so as to facilitate closure of distal port section 830 (i.e., via endotracheal tube string 806) and flexible detachable film inlet 854 (i.e., via drawstring 808), in order to substantially contain and seal biological material (e.g., secretions, pathogens, microorganisms), to which inner surface 828 and flexible detachable film layer 850 have been exposed to, within each of their respective substantially enclosed internal volumes. Accordingly, with reference to FIG. 8B, when endotracheal tube string 806 is pulled (via proximal end 822) to a certain extent so as to increase its tension, rim 832 and string distal section 836 detach from inner surface 820 and distal end 824 is constricted so as to form constriction 838. In this operative state (FIG. 8B) distal port section 830 is partially closed and drawstring ends 856 are in a slack state.

According to another operative state of multi-layer endotracheal tube apparatus 800, shown in FIG. 8C, endotracheal tube string 806 is pulled further (i.e., string tension thereof increases), thereby substantially closing distal port section 830, as well as constricting flexible elongated inner tube layer 804 so as to decrease its enclosed internal volume thereof. This reduction of its internal volume lowers the area of contact with inner surface 820 thus substantially reducing friction as flexible elongated inner tube layer 804 is removed from within outer flexible elongated tube layer 802. At this illustrative operative state, endotracheal tube string 806 draws drawstring ends 856 tighter, thus partially detaching flexible detachable film inlet 854 from circumferential coupling with peripheral edge 848 as well as further drawing flexible detachable film inlet 854 toward closure.

FIG. 8D illustrates a further operative state of multi-layer endotracheal tube apparatus 800, where endotracheal tube string 806 is pulled along with substantial part of flexible elongated inner tube layer 804 via proximal end 822. In this operative state, the tension in drawstring 808 draws flexible detachable film inlet 854 to close so as to form a substantially closed volume (e.g., as in a closed sack, sachet, analogous to the ubiquitous plastic garbage disposal bag, and the like) and complete detachment from peripheral edge 848. Biological material accumulated on the exposed surface of flexible detachable film layer 850, as well as inner surface 828 (FIG. 8A) of flexible elongated inner tube layer 804 are substantially sealed within their respective formed closed volumes, such that substantially no biological material seeps therefrom during their withdrawal and complete removal from within outer flexible elongated tube layer 802. Consequently, each flexible elongated inner tube layer 804 is associated (i.e., paired) with a respective flexible detachable film layer 850 so when such a pair is removed, a new successive pair is revealed underneath, whereupon the process is repeated until all remaining pairs expire.

It will be appreciated by persons skilled in the art that the disclosed technique is not limited to what has been particularly shown and described hereinabove. Rather the scope of the disclosed technique is defined only by the claims, which follow.

1. Multi-layer endotracheal tube apparatus comprising:
an outer flexible elongated tube layer having a proximal end and a distal end, defining an inner surface therebetween;
at least one flexible elongated inner tube layer having a proximal end and a distal end, defining an inner surface, an outer surface, and an internal volume therebetween, said distal end of said at least one flexible elongated inner tube layer defining a distal port section, said at least one flexible elongated inner tube layer extending substantially within and at least partially along inner length of said outer flexible elongated tube layer, said distal end of said at least one flexible elongated inner tube layer detachably coupled along an inner substantially closed circumference of said inner surface of said outer flexible elongated tube layer; and
at least one closure mechanism at least partially located in an area of said distal end of said at least one flexible elongated inner tube layer, said at least one closure mechanism operative to close said distal port section, and detach said distal end of said at least one flexible elongated inner tube layer from said inner surface of said outer flexible elongated tube layer, as to enable withdrawal of said at least one flexible elongated inner tube layer via said proximal end of said outer flexible elongated tube layer.

2. The multi-layer endotracheal tube apparatus according to claim 1, wherein said distal port section is terminated by a rim, said rim being detachably coupled along an inner substantially closed circumference of said inner surface of said outer flexible elongated tube layer.

3. The multi-layer endotracheal tube apparatus according to claim 1, wherein said at least one closure mechanism comprises a string having a string proximal section and a string distal section, said string extending at least partially substantially along length of said at least one flexible elongated inner tube layer, said string proximal section winds at least partially about said distal port section for constricting said distal end of said at least one flexible elongated inner tube layer such to substantially close said distal port section, when said string proximal section is pulled.

4. The multi-layer endotracheal tube apparatus according to claim 3, wherein said string substantially closes said distal port section prior to said detachment of said distal end of said at least one flexible elongated inner tube layer from said inner surface of said outer flexible elongated tube layer.

5. (canceled)

6. The multi-layer endotracheal tube apparatus according to claim 3, wherein said string distal section has a string distal end detachably coupled with said inner surface of said outer flexible elongated tube layer.

7. The multi-layer endotracheal tube apparatus according to claim 3, wherein said string is wound substantially helically around said outer surface of said at least one flexible elongated inner tube layer, substantially along said outer length thereof, for at least partially decreasing said internal volume.

8. (canceled)

9. The multi-layer endotracheal tube apparatus according to claim 1, further comprising a withdrawal string coupled to said proximal end of said at least one flexible elongated inner tube layer for facilitating removal of said at least one flexible elongated inner tube layer from within said outer flexible elongated tube layer.

10. (canceled)

11. The multi-layer endotracheal tube apparatus according to claim 1, wherein said at least one closure mechanism is operative to substantially contain biological material, substantially within said at least one flexible elongated inner tube layer during said withdrawal.

12. The multi-layer endotracheal tube apparatus according to claim 1, wherein said at least one flexible elongated inner tube layer is collapsible.

13. (canceled)

14. The multi-layer endotracheal tube apparatus according to claim 3, wherein said string, which said winds about said distal port section, forms a knot.

15. The multi-layer endotracheal tube apparatus according to claim 1, wherein said closure mechanism comprises an inflatable body coupled to either one of said distal end of said at least one flexible elongated inner tube layer and said distal end of said inner surface of said outer flexible elongated tube layer, for substantially closing said distal port section when said inflatable body is in an inflated state.

16. The multi-layer endotracheal tube apparatus according to claim 1, wherein said at least one closure mechanism comprises a closed inflatable volume incorporated at said distal end of said at least one flexible elongated inner tube layer.

17. The multi-layer endotracheal tube apparatus according to claim 1, wherein said at least one closure mechanism is implemented by said distal port section being constructed to crumple thereby sealing said distal port section.

18. The multi-layer endotracheal tube apparatus according to claim 1, wherein said at least one closure mechanism comprises a flexible elongated rod, which substantially extends within and substantially along length of said outer flexible tube layer, said flexible elongated rod having a rod distal end coupled with either one of said inner surface and said outer surface of said at least one flexible elongated inner tube layer at said distal end thereof, said flexible elongated rod constricts said distal port section when said flexible elongated rod is rotated, thereby substantially closing said distal port section.

19. The multi-layer endotracheal tube apparatus according to claim 1, wherein said at least one flexible elongated inner tube layer is constructed from a flexible rigid tube section that includes said proximal end of said at least one flexible elongated inner tube layer and a twistable tube section that includes said distal end of said at least one flexible elongated inner tube layer, said flexible rigid tube section and said twistable tube section are circumferentially coupled to each other substantially at a neighborhood of said distal end of said at least one flexible elongated inner tube layer, said twistable tube section having substantially lesser of an ability to resist deformation by an applied torque than that of said flexible rigid tube section, wherein said at least one closure mechanism is implemented by when said twistable tube section twists and deforms to form a constriction in response to said applied torque thereby substantially closing said distal port section.

20. The multi-layer endotracheal tube apparatus according to claim 19, wherein after said closing of said distal port section, further applied torque causes said distal end of said at least one flexible elongated inner tube layer to said detach from said inner surface of said outer flexible elongated tube layer.

21. The multi-layer endotracheal tube apparatus according to claim 3, further comprising:
a laryngeal mask portion coupled with said outer flexible elongated tube layer at said distal end thereof, said laryngeal mask portion includes an inflatable laryngeal cuff having an outer periphery and an inner periphery that defines an opening, said opening defines a peripheral edge;
at least one flexible detachable film layer, said at least one flexible detachable film layer having a rim, said rim defines a flexible detachable film inlet that is detachably and circumferentially coupled with said peripheral edge, said at least one flexible elongated inner tube layer in communication with said opening, said at least one flexible detachable film layer detachably covers said inner periphery so as to substantially follow the circumference of said peripheral edge;
wherein said at least one closure mechanism further includes at least one other closure mechanism operative to close said flexible detachable film inlet, and detach said at least one flexible detachable film layer from said inner periphery so as to enable withdrawal of said at least one flexible detachable film layer, via said proximal end of said outer flexible elongated tube layer.

22. The multi-layer endotracheal tube apparatus according to claim 21, wherein said at least one other closure mechanism comprises a drawstring having drawstring ends, said drawstring ends coupled with either one of said string and said at least one flexible elongated inner tube layer, said drawstring at least partially extending and substantially circumferentially coupled along perimeter of said rim for constricting and substantially closing said flexible detachable film inlet to form a substantially closed volume, when said string proximal section is substantially pulled.

23. The multi-layer endotracheal tube apparatus according to claim 1, wherein said at least one flexible elongated inner tube layer is paired with a respective one of said at least one closure mechanism, such that said withdrawal of said at least one flexible elongated inner tube layer withdraws said respective one of said at least one closure mechanism.

24. Method for removal of an inner layer from within a multi-layer endotracheal tube, and material substantially contained within a volume defined by the inner layer, the multi-layer endotracheal tube including an outer flexible elongated tube layer, at least one flexible elongated inner tube layer and a closure mechanism, the outer flexible elongated tube layer having a proximal end and distal end, defining an inner surface therebetween, the flexible elongated inner tube layer having a proximal end and a distal end that defines a distal port section, the flexible elongated inner tube layer extending substantially within and at least partially along inner length of the outer flexible elongated tube layer, the distal end of the flexible elongated inner tube layer detachably coupled along an inner substantially closed circumference of the inner surface of the outer flexible elongated tube layer, the closure mechanism at least partially located in an area of the distal end of the flexible elongated inner tube layer, the method comprising the procedures of:
closing said distal port section by said closure mechanism;
detaching said distal end of said flexible elongated inner tube layer from said inner surface of said outer flexible elongated tube layer; and
removing said flexible elongated inner tube layer from within said outer flexible elongated tube layer via said proximal end of said outer flexible elongated tube layer.

25. (canceled)

26. The method according to claim 24, wherein procedure of said closing substantially precedes procedure of said detaching.

27. The method according to claim 24, wherein procedure of said detaching precedes procedure of said removing.

28. Multi-layer endotracheal tube apparatus comprising:
an outer flexible elongated tube layer having a proximal end and a distal end, defining an inner surface therebetween; and
at least one flexible inner tube layer having a proximal end and a distal end, at least part of said at least one flexible inner tube layer is layered onto said inner surface of said outer flexible tube layer, said at least one flexible inner tube layer extending substantially within and at least partially along inner length of said outer flexible elongated tube layer, said at least one flexible inner tube layer is operative to detach from within said outer flexible elongated inner tube layer, as to enable withdrawal of said at least one flexible inner tube layer via said proximal end of said outer flexible elongated tube layer, wherein said at least one flexible inner tube layer is operative to said detach from another one of said at least one flexible inner tube layer.

29-33. (canceled)

34. The multi-layer endotracheal tube apparatus according to claim 28, further comprising a string having a string proximal section and a string distal section, said string extending at least partially substantially along length of said at least one flexible inner tube layer, said string proximal section coupled with said distal end of said at least one flexible inner tube layer, said distal end of said at least one flexible inner tube layer is detachably coupled along an inner substantially closed circumference of said inner surface of said outer flexible elongated tube layer, said at least one flexible inner tube layer is operative to detach from within said outer flexible elongated inner tube layer such that said distal end of said at least one flexible inner tube layer folds upon remaining portion of said at least one flexible inner tube layer when said string proximal section is pulled, so as to enable said withdrawal.

35. The multi-layer endotracheal tube apparatus according to claim 24, wherein said at least one flexible inner tube layer has an inner surface and an outer surface, said inner surface of said at least one flexible inner tube layer substantially does not contact said inner surface of said outer flexible elongated inner tube layer and said inner surface of another one of said at least one flexible inner tube layer during said withdrawal.

36. Multi-layer endotracheal tube apparatus comprising:
an outer flexible elongated tube layer having a proximal end and a distal end, defining an inner surface and an outer surface therebetween; and
a flexible elongated layer having an engaging surface and an exposure surface, said flexible elongated layer extends substantially from said proximal end and substantially along said outer surface toward said distal end where said flexible elongated layer continues to extend into said outer flexible elongated tube layer substantially along said inner surface toward said proximal end, so as to enable withdrawal of at least part of said flexible elongated layer via said proximal end, such that only said engaging surface of said flexible elongated layer is in contact with said inner surface and said outer surface.
